(19) United States
(12) Patent Application Publication
(10) Pub. No.: US 2026/0053727 A1
(43) Pub. Date: Feb. 26, 2026

(54) PERSONAL CARE COMPOSITIONS WITH ENZYME-MODIFIED OILS (EMOS)

(71) Applicant: GLYCOSBIO INC., Houston, TX (US)

(72) Inventors: Daniel J. MONTICELLO, The Woodlands, TX (US); Nathaniel Scott SCHOCKER, Pearland, TX (US)

(73) Assignee: GLYCOSBIO INC., Houston, TX (US)

(21) Appl. No.: 19/361,239

(22) Filed: Oct. 17, 2025

Related U.S. Application Data
(63) Continuation of application No. PCT/US2024/025563, filed on Apr. 19, 2024.
(60) Provisional application No. 63/497,630, filed on Apr. 21, 2023.

(51) Int. Cl. A61K 8/92; A61Q 17/00; A61Q 19/10

(52) U.S. Cl. CPC A61K 8/922; A61Q 17/005; A61Q 19/10

(57) ABSTRACT

Compositions including an oil in an amount of about 0.25% to about 10% by weight of the total weight of the composition are provided. The oil includes free fatty acids derived from a first oil source, and the first oil source includes a fatty acid profile with at least 50% caprylic acid, capric acid, or a combination thereof. Methods of making and using such compositions are also provided.

### CROSS-REFERENCE TO RELATED APPLICATIONS

This application is a continuation of PCT/US24/25563, filed Apr. 19, 2024, which claims priority to U.S. Provisional Patent Application No. 63/497,630, filed Apr. 21, 2023, both of which are incorporated herein by reference in their entirety.

### STATEMENT OF GOVERNMENT INTEREST

Not applicable.

### INCORPORATION BY REFERENCE

Methods for producing enzyme modified oils (EMOs) and/or free fatty acid (FFA) oils, and properties thereof, are disclosed in International Patent Application Nos. PCT/US2018/055583, filed Oct. 12, 2018, published as WO2019/075307, PCT/US2020/014182, filed Jan. 17, 2020, published as WO2020/150661, PCT/US2022/024709, filed Apr. 13, 2022, published as WO2022/221470, PCT/US2022/024711, filed Apr. 13, 2022, published as WO2022/221472, and U.S. Patent Application Publication No. 2020/0146307 (U.S. Ser. No. 16/746,700), each of which is incorporated herein by reference in its entirety.

### BACKGROUND

Consumers desire personal care compositions, such as body, face washes, shampoos, and hand soaps, with naturally-derived and/or minimally processed ingredients because they are safer for humans and animals as well as the environment. Cleansing personal care compositions include cleansing surfactants and antimicrobial compositions, which are commonly synthetic chemicals that are harsh on the human body. The cleansing surfactants remove undesirable soil, particles, and fatty matter from the skin and/or hair, and antimicrobial compositions kill, stop, or mitigate microorganism growth on the skin and/or hair, or within the personal care composition itself. Although some natural free fatty acids (FFAs) have some antimicrobial activity, only a fraction of types of FFAs have such bioactivity, the said activity is weak and requires high effective compound concentrations, appropriate conditions (e.g. pH of the formulation), and many bacteria are resistant to antibacterial effects of FFAs. Thus, there is a need for personal care compositions with naturally-derived and/or minimally processed ingredients, including antimicrobial compositions with FFAs.

### SUMMARY

The present disclosure provides personal care compositions and methods of making and using the same. The present disclosure further provides antimicrobial compositions and methods of making and using the same.

In some embodiments, compositions include an oil in an amount of about 0.25% to about 5% by weight of the total weight of the composition are provided. The oil includes free fatty acids derived from a first oil source, and the first oil source includes a fatty acid profile with at least 50% caprylic acid, capric acid, or a combination thereof.

In some embodiments, the oil further includes free fatty acids derived from a second oil source. The second oil source includes a fatty acid profile that is different from the fatty acid profile of the first oil source.

In some embodiments, compositions include a surfactant and an oil in an amount of about 0.25% to about 5% by weight of the total weight of the composition. The oil includes free fatty acids derived from a first oil source, and the first oil source includes a fatty acid profile comprising at least 50% caprylic acid, capric acid, or a combination thereof.

In some embodiments, methods for making compositions include adding an oil to the compositions in an amount of about 0.25% to about 5% by weight of the total weight of the composition. The oil includes free fatty acids derived from a first oil source, and the first oil source includes a fatty acid profile with at least 50% caprylic acid, capric acid, or a combination thereof. The oil further includes free fatty acids derived from a second oil source, and the second oil source includes a fatty acid profile that is different from the fatty acid profile of the first oil source.

In some embodiments, methods for making compositions include adding a surfactant to the compositions, and further adding an oil to the compositions in an amount of about 0.25% to about 5% by weight of the total weight of the compositions. The oil includes free fatty acids derived from a first oil source, and the first oil source includes a fatty acid profile with at least 50% caprylic acid, capric acid, or a combination thereof.

In some embodiments, methods for making compositions include hydrolyzing an oil from a first oil source to provide free fatty acids and glycerol, and further separating a fraction enriched in caprylic acid, capric acid, or a combination thereof from the glycerol and remaining free fatty acids. The separating caprylic acid, capric acid, or a combination thereof includes a winterization process.

### BRIEF DESCRIPTION OF THE FIGURES

FIG. 1 depicts a method for making Fractionated Coconut Oil (FCO), a caprylic/capric-enriched TriAcylGlycerol (TAG) acid used to make an antimicrobial composition.

FIG. 2 depicts a method to make FCO FFA, a caprylic/capric acid-enriched free fatty acid (FFA) oil produced from FCO. The terms FCO FFA and medium chain triglyceride (MCT) FFA are use interchangeably in this document.

FIG. 3 depicts a method to make Enzyme Modified MCT FFA (EM-MCT FFA, a caprylic/capric acid-enriched free fatty acid (FFA) oil produced directly from coconut oil.

FIG. 4A depicts a 2-minute bactericidal assay with additional essential oil (“Cassia”) or flax antimicrobial oil (Enzyme Modified Oil, EMO) compositions.

FIG. 4B depicts the MCT EMO or FFA effect on E. coli, in particular 2-min bactericidal action.

FIG. 5 depicts a 2-minute bactericidal assay with 5 mg/mL antimicrobial EMO against E coli and S. aureus.

FIG. 6 depicts bactericidal comparison of C8:0 FFA, MCT FFA, and coconut FFA against E coli.

FIG. 7A depicts a body Dettol body wash (DBW) with and without MCT FFA at various concentrations.

FIG. 7B depicts a body Dettol body wash (DBW) with and without MCT FFA at various concentrations.

FIG. 8 depicts a 2-minute bactericidal assay with body wash alone or body wash with 1% FCO FFA.

FIG. 9 depicts formulations 1-4 from Table 1 in 2-minute bactericidal assay with E. coli.

FIG. 10 depicts antimicrobial test EN 1276 with Dettol body wash (DBW) and E. coli, with 8:1:1 ratios of bodywash:interfering substance:bacterial solution, for 2-minutes.

FIG. 11 depicts a bactericidal assay with body wash alone or body wash plus 0.5% FCO FFA, with ratios of 4:5:1 bodywash:phosphate-buffered saline (PBS)/bacterial solution, for 2-minutes.

FIG. 12 depicts results with initial GlycosBio body wash (GBW) formulations from Table 2.

FIG. 13 depicts an ingredient screen with 1% FCO FFA.

FIG. 14 depicts a bactericidal assay with formulations from Table 4 with or without sodium lactate.

FIG. 15A depicts a bactericidal assay with excess FCO FFA added.

FIG. 15B depicts a bactericidal assay with excess FCO FFA added.

FIG. 16 depicts results of Table 5 Formulations against E. coli.

FIG. 17 depicts a multi-day test of GBW formulations with or without 1% additional FCO FFA added.

FIG. 18 depicts sodium lauryl sulfate (SLS) and FCO FFA concentrations in GBW formulations against E. coli.

FIG. 19 depicts an 8-day test of 090622 formulations and a 2-day test of increased SLS/FFA formulations against S. aureus.

FIG. 20 depicts a 4-day test of 091222 formulations and 0-day test of Table 6 formulation with increased SLS/FFA and all salts against E. coli and S. aureus.

FIG. 21 depicts an assay of Table 8 formulations against S. aureus.

FIG. 22 depicts an assay of Table 9 formulations against S. aureus.

FIG. 23 depicts and assay of Table 8 formulations against E. coli and S. aureus 27217 1-week post-formulation.

FIG. 24 depicts membrane permeability of E. coli caused by antimicrobial nanoemulsions as measured by Fluorescence increase with propidium iodide.

FIG. 25 depicts membrane potential of E coli caused by antimicrobial nanoemulsions, as measured by Fluorescence increase with DiOC2.

FIG. 26 depicts membrane potential of E coli caused by antimicrobial nanoemulsions, as measured by Fluorescence increase with DiSC3.

FIG. 27 depicts a graph of free fatty acids in coconut oil.

FIG. 28 depicts the equilibrium between acids and their sodium salts.

FIG. 29 depicts the effects of nCo/Fx-25 on Staph species at two pHs.

FIG. 30 depicts the bactericidal effect of 2% and 4% Activated Oil (AO) formulated into a commercial body wash at various pHs.

FIG. 31 depicts the bactericidal effect of 2% and 4% Activated Oil (AO) formulated into a commercial body wash at various pHs.

### DETAILED DESCRIPTION

The present disclosure further provides personal care compositions and methods of making and using the same. The present disclosure further provides antimicrobial compositions and methods of making and using the same.

### Definitions

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As used herein, “about” refers to an amount within +/−10% of a recited amount. Further, “about” therefore means+/−10, 9, 8, 7, 6, 5, 4, 3, 2, or 1 of a recited amount. By way of example, but not limitation, where a component is recited as “about 10,” this would include from 9 to 11.

As used herein, a “therapeutically effective amount” refers to an amount of a composition of the present disclosure effective yield a desired therapeutic response. By way of example, but not limitation, an amount effective to delay the growth of a microorganism can be “therapeutically effective amount.” The specific therapeutically effective amount will vary with such factors as the particular condition being treated, the physical condition of the subject, the type of subject being treated, the duration of the treatment, the nature of concurrent therapy (if any), and the specific formulations employed and the structure of the composition.

As used herein, “free fatty acid (FFA)” refers to a non-esterified fatty acid in its carboxylic acid form or carboxylate salt form.

As used herein, a “monoacylglycerol (MAG),” also known as a monoglyceride, is a glyceride consisting of one fatty acid chain covalently bonded to a glycerol molecule through an ester linkage.

As used herein, a “diacylglycerol (DAG),” also known as a diglyceride, is a glyceride consisting of two fatty acid chains covalently bonded to a glycerol molecule through ester linkages.

As used herein, a “triacylglycerol (TAG),” also known as a triglyceride, is a glyceride consisting of three fatty acid chains covalently bonded to a glycerol molecule through ester linkages. TAGs may also be classified as having a long or medium chain length. Long chain TAGs contain fatty acids with 14 or more carbons, while medium chain TAGs contain fatty acids with 6 to 12 carbons, 8 to 10 carbons, or about or any range from 6, 7, 8, 9, 10, 11, and 12 carbons. Long chain TAGs can include omega-3 and omega-6 fatty acids. Medium chain TAGs have saturated fatty acids and thus do not contain omega-3 or omega-6 fatty acids. Long chain TAGs (LCT) and medium chain triglycerides (MCT) can serve as energy sources.

As used herein, “free fatty acid oil (FFA oil)” or “fatty acid oil” refers to an oil that includes free fatty acids that result from the chemical, physical or enzymatic hydrolysis of triacylglycerides (TAGs) in a starting oil source(s) to FFA and glycerol, with subsequent removal of the glycerol. It should be understood that the FFA oil can include some glycerol and other components of the source TAG oil, which may be dissolved in the FFA oil.

As used herein, “enzyme-modified oil (EMO)” refers to an oil that includes MAGs and, optionally DAGs, and/or FFA that results from enzymatic hydrolysis of a starting oil source(s) to yield FFA and glycerol, followed by separation of the glycerol, and at least partial enzymatic re-esterification of glycerol with the FFA to yield MAGs and, optionally some DAGs, and, in some instances, some residual FFAs. Unless otherwise stated herein, an EMO should be understood to be substantially free of TAGs (below a detectable level). In some embodiments, an EMO can have less than 5%, less than 4%, less than 3%, less than 2%, or less than 1% TAGs.

As used herein, a “subject” refers to any living being, such as a human being or animal.

As used herein, “surfactant” or “emulsifier” refers to a substance that reduces the surface tension between two liquids or, in the case of a solid lipid nanoemulsion, between the liquid phase and the solid lipid phase. Surfactants, or surface-active agents, are compounds that lower the surface tension between two liquids or between a liquid and a solid. An emulsifier is a surfactant that stabilizes emulsions. Emulsifiers coat droplets within an emulsion and prevent them from coming together, or coalescing.

Surfactants are commonly used as emulsifiers as they are known to stabilize emulsions. By way of example, but not limitation, surfactants (and emulsifiers) can include polysorbates, phospholipids, sterols, cationic lipids, poloxamers, sorbitan esters, sugar esters, polyoxyethylene conjugates, ammonium phosphatidyl lapsidate, polyphenols, polyvinylalcohol, lecithin, ethanol, lysophospholipids, ceramides, Tweens, Spans, Kolliphor, propylene glycol, vitamin D, polyethylene glycol, polyethylene glycol derivatives, pectin, gum acacia, modified gum acacia, agar, ghatti gum, modified ghatti gum, pectin, carrageenan, xanthan gum, modified starches, modified alginate, fatty alcohols, and ethoxylated polyols.

In some embodiments, compositions include an oil in an amount of about 0.25% to about 5% by weight of the total weight of the composition are provided. The oil includes free fatty acids derived from a first oil source, and the first oil source includes a fatty acid profile with at least 50% caprylic acid, capric acid, or a combination thereof.

In some embodiments, the oil may further include free fatty acids derived from a second oil source. The second oil source includes a fatty acid profile that is different from the fatty acid profile of the first oil source, or alternatively, is the same as the type of oil as the first oil source.

In some embodiments, compositions include a surfactant, and further an oil in an amount of about 0.25% to about 5% by weight of the total weight of the composition. The oil includes free fatty acids derived from a first oil source, and the first oil source includes a fatty acid profile comprising at least 50% caprylic acid, capric acid, or a combination thereof.

### Oil

The oil described herein may be included as a component of a product or an composition, such as products and compositions used for personal care (e.g., soaps, body washes, hand soaps, etc. and may provide an enhanced antimicrobial property on the product or composition used in as well as other benefits. It should be understood that these properties may be present in any of the embodiments described herein below.

In any of the embodiments, the product or composition can include the oil component in an amount from about 0.25% to about 10% by weight out of the total weight of the composition. By way of example, but not limitation, the product or composition can include the oil component in an amount from about 0.25% to about 4.5%, about 0.25% to about 4%, about 0.25% to about 3.5%, about 0.25% to about 3%, about 0.25% to about 2.5%, about 0.25% to about 2%, about 0.25% to about 1.5%, about 0.25% to about 1%, about 0.25% to about 0.75%, about 0.25% to about 0.5%, about 0.5% to about 5%, about 0.5% to about 4.5%, about 0.5% to about 4%, about 0.5% to about 3.5%, about 0.5% to about 3%, about 0.5% to about 2.5%, about 0.5% to about 2%, about 0.5% to about 1.5%, about 0.5% to about 1%, about 0.5% to about 0.75%, about 0.75% to about 5%, about 0.75% to about 4.5%, about 0.75% to about 4%, about 0.75% to about 3.5%, about 0.75% to about 3%, about 0.75% to about 2.5%, about 0.75% to about 2%, about 0.75% to about 1.5%, about 0.75% to about 1%, about 1% to about 5%, about 1% to about 4.5%, about 1% to about 4%, about 1% to about 3.5%, about 1% to about 3%, about 1% to about 2.5%, about 1% to about 2%, about 1% to about 1.5%, about 1.5% to about 5%, about 1.5% to about 4.5%, about 1.5% to about 4%, about 1.5% to about 3.5%, about 1.5% to about 3%, about 1.5% to about 2.5%, about 1.5% to about 2%, about 1.75% to about 5%, about 1.75% to about 4.5%, about 1.75% to about 4%, about 1.75% to about 3.5%, about 1.75% to about 3%, about 1.75% to about 2.5%, about 1.75% to about 2%, about 2% to about 5%, about 2% to about 4.5%, about 2% to about 4%, about 2% to about 3.5%, about 2% to about 3%, about 2% to about 2.5%, about 2.25% to about 5%, about 2.25% to about 4.5%, about 2.25% to about 4%, about 2.25% to about 3.5%, about 2.25% to about 3%, about 2.25% to about 2.5%, about 2.5% to about 5%, about 2.5% to about 4.5%, about 2.5% to about 4%, about 2.5% to about 3.5%, about 2.5% to about 3%, about 2.75% to about 5%, about 2.75% to about 4.5%, about 2.75% to about 4%, about 2.75% to about 3.5%, about 2.75% to about 3%, about 3% to about 5%, about 3% to about 4.5%, about 3% to about 4%, about 3% to about 3.5%, about 3.25% to about 5%, about 3.25% to about 4.5%, about 3.25% to about 4%, about 3.25% to about 3.5%, about 3.5% to about 5%, about 3.5% to about 4.5%, about 3.5% to about 4%, about 3.75% to about 5%, about 3.75% to about 4.5%, about 3.75% to about 4%, about 4% to about 5%, about 4% to about 4.5%, about 4.25% to about 5%, about 4.25% to about 4.5%, about 4.75% to about 5%, about 0.25%, 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10% or any range or value therebetween by weight out of the total weight of the composition.

In any of the foregoing embodiments, the oil can include a blend of two or more processed oils, from each of a first oil source and a second oil source, and optionally, a third oil source, a fourth oil source, or more oil sources. By way of example, but not limitation, the oil component can include a blend of two, three, four or more processed oils.

In any of the foregoing embodiments, the oil includes FFA derived from a first oil source with a fatty acid profile including caprylic acid, capric acid, or a combination thereof, wherein the amount of caprylic acid, capric acid, or a combination thereof within the first oil source is at least 50%. By way of example, but not limitation, the oil includes FFA derived from a first oil source has a fatty acid profile that includes caprylic acid, capric acid, or a combination thereof in an amount of at least 50%, at least 55%, at least 60%, at least 65%, at least 70%, at least 75%, at least 80%, at least 85%, at least 90%, at least 95%, at least 99%, at least 99.9%, or 100% caprylic acid, capric acid, or a combination thereof. By way of example, but not limitation, the first oil source has a fatty acid profile that includes about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, about 95% to about 100%, about 50% to about 99%, about 55% to about 99%, about 60% to about 99%, about 65% to about 99%, about 70% to about 99%, about 75% to about 99%, about 80% to about 99%, about 85% to about 99%, about 90% to about 99%, about 95% to about 99%, about 50% to about 90%, about 55% to about 90%, about 60% to about 90%, about 65% to about 90%, about 70% to about 90%, about 75% to about 90%, about 80% to about 90%, about 85% to about 90%, about 50% to about 85%, about 55% to about 85%, about 60% to about 85%, about 65% to about 85%, about 70% to about 85%, about 75% to about 85%, about 80% to about 85%, about 50% to about 80%, about 55% to about 80%, about 60% to about 80%, about 65% to about 80%, about 70% to about 80%, about 75% to about 80%, about 50% to about 75%, about 55% to about 75%, about 60% to about 75%, about 65% to about 75%, about 70% to about 75%, about 50% to about 70%, about 55% to about 70%, about 60% to about 70%, about 65% to about 70%, about 50%, about 55%, about 60%, about 65%, about 70%, about 75%, about 80%, about 85%, about 90%, about 95%, about 96%, about 97%, about 98%, about 99%, about 100%, or any range or value therebetween of the total fatty acid content.

In any of the foregoing embodiments, the oil comprises FFA derived from a first oil source comprising a ratio of C8:C10 fatty acids of about 30:70, of about 40:60, of about 50:50, of about 60:40, or of about 70:30, wherein in any of the above embodiments, the oil source has less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2% or less than about 1% of C12-C18 fatty acids. In these and any of the foregoing embodiments, the first oil source is fractionated coconut oil.

In any of the foregoing embodiments, the FFA derived from the first oil source are from about 10% to about 100% by weight of the total weight of the oil. By way of example, but not limitation, FFA derived from the first oil source are at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or 100% by weight of the total weight of the oil. By way of further example, but not limitation, the oil can include the FFA from the first oil source in an amount about 10% to about 100%, about 10% to about 99%, about 15% to about 99%, about 20% to about 99%, about 25% to about 99%, about 30% to about 99%, about 35% to about 99%, about 40% to about 99%, about 50% to about 99%, about 60% to about 99%, about 70% to about 99%, about 80% to about 99%, about 90% to about 99%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 90%, about 25% to about 80%, about 25% to about 75%, about 25% to about 70%, about 25% to about 60%, about 25% to about 50%, about 25% to about 40%, about 25% to about 30%, about 30% to about 90%, about 30% to about 80%, about 30% to about 75%, about 30% to about 70%, about 30% to about 60%, about 30% to about 50%, about 30% to about 40%, about 40% to about 90%, about 40% to about 80%, about 40% to about 75%, about 40% to about 70%, about 40% to about 60%, about 40% to about 50%, about 50% to about 90%, about 50% to about 80%, about 50% to about 75%, about 50% to about 70%, about 50% to about 60%, about 60% to about 90%, about 60% to about 80%, about 60% to about 75%, about 60% to about 70%, about 70% to about 90%, about 70% to about 80%, about 80% to about 90%, about 85% to about 90%, about 10%, about 15%, about 20%, about 25%, about 30%, about 40%, about 50%, about 60%, about 70%, about 75%, about 80%, about 90%, about 95%, about 99%, 100%, or any range or value therebetween by weight of the total weight of the oil.

In any of the foregoing embodiments, the FFA derived from the first oil source can be derived from an oil source(s) selected from a plant, an animal, a fish, an algal oil, and combinations thereof that contains some amount of capric and/or caprylic acids. By way of example but not limitation, the FFA derived from the first oil source is coconut oil that has been processed and enriched in caprylic acid, capric acid, or a combination thereof. For example, the first oil source can be a fractionated coconut oil (FCO) or a medium chain triglyceride (MCT) oil.

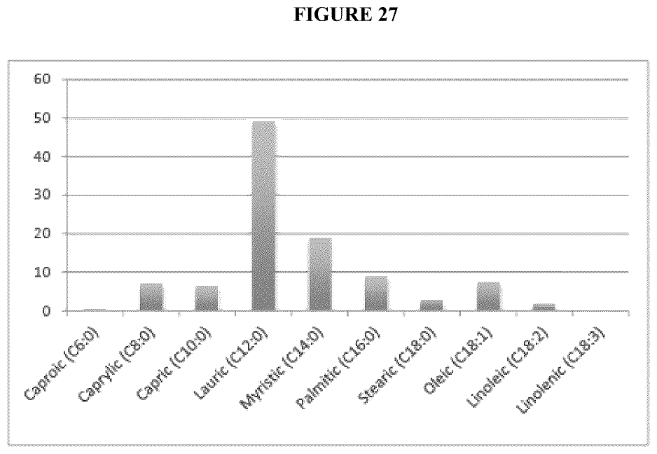

Table A below and FIG. 27 show non-limiting examples of natural coconut oil FFA and FCO. As shown, caprylic acid and capric acid make up a small percentage of the FFA of coconut oil. On the other hand, lauric acid makes up about 50% of the total coconut oil fatty acids and can be used to make the synthetic detergents sodium lauryl sulphate and sodium laureth sulfate.

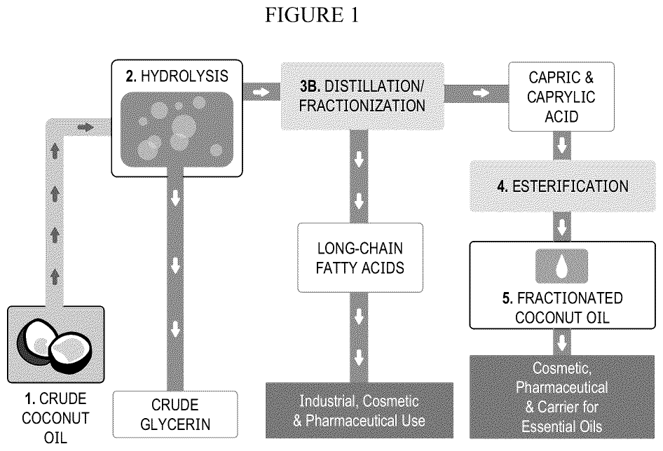

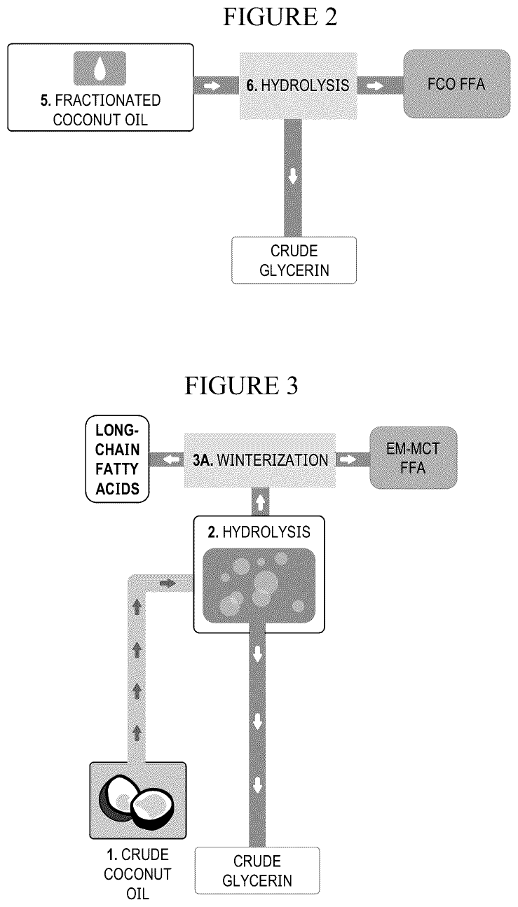

FIG. 1 illustrates some options for producing a FFA oil enriched with caprylic and capric acids. FCA FFA can be produced from FCO. FCO is produced through the chemical, physical or enzymatic hydrolysis of coconut oil to produce a mixture of the free fatty acids depicted in Table A and FIG. 27. Following this hydrolysis, the FFAs are fractionated by molecular distillation (FIG. 1, Step 3B). This separates the caprylic and capric acids (and other lower boiling point molecules) from the lauric and other long-chain fatty acids. This “MCT” fraction containing the caprylic and capric acids is reacted with glycerol to produce TriAcylGlycerols (TAGs). This new oil is FCO. FCO FFA (aka MCT FFA) can be produced from this oil by subsequent hydrolysis back to the FFA and glycerol (FIG. 2).

An alternative, more natural approach to producing a caprylic/capric-rich FFA oil is depicted in FIG. 3. In this case, the coconut oil is combined with an appropriate lipase (such as Amano Lipase AY) and a buffered solution. The TAGs are enzymatically hydrolyzed to FFA, then the solution is heated (to ˜70° C.) to inactivate the. The FFA oil is separated from the aqueous phase and slowly cooled to between 37° C. and 43° C. in order to crystalize the longer chain fatty acids. These are then removed by filtration or centrifugation. This separation technique is generically known as “winterization” although it is not usually applied to fatty acids. This enzymatically produced material is Enzyme Modified MCT FFA (EM-MCT FFA) to produce TAGs.

Thus, in any one or more of the foregoing embodiments, methods for making compositions include hydrolyzing an oil from a first oil source to provide free fatty acids and glycerol, and further separating a fraction enriched in caprylic acid, capric acid, or a combination thereof from the glycerol and remaining free fatty acids. The separating caprylic acid, capric acid, or a combination thereof includes a winterization process. In some embodiments, the method further includes isolating a fraction comprising the caprylic acid, the capric acid, or the combination thereof.

TABLE A
Fatty acid content of typical Coconut
oil and Fractionated Coconut oil.

| | Virgin | |
|---|---|---|
| | Coconut | Fractionated |
| Fatty Acids | Oil | Coconut Oil |
| Caproic (C6:0) | 0.4 | 0.1 |
| Caprylic (C8:0) | 7.8 | 41.3 |
| Capric (C10:0) | 6.7 | 38.4 |
| Lauric (C12:0) | 49.5 | 11.5 |
| Myristic (C14:0) | 18.1 | 4.5 |
| Palmitic (C16:0) | 8.2 | 2.1 |
| Stearic (C18:0) | 2.2 | 0.8 |
| Arachidic (C20:0) | 0.1 | 0 |
| Oleic (C18:1) | 5.6 | 1.2 |
| Linoleic (C18:2) | 1.4 | 0.1 |

In any of the foregoing embodiments, the composition may further include FFA derived from a second oil source with a fatty acid profile that is different from the fatty acid profile of the first oil source. The FFA derived from the second oil source are from about 1% to about 90% by weight of the total weight of the oil. By way of example, but not limitation, FFA derived from the second oil source are at least 1%, at least 2%, at least 3%, at least 4%, at least 5%, at least 6%, at least 7%, at least 8%, at least 9%, least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% by weight of the total weight of the oil. By way of further example, but not limitation, the oil can include FFA derived from the second oil source in an amount that is about 1% to about 90%, about 5% to about 90%, about 10% to about 90%, about 15% to about 90%, about 20% to about 90%, about 25% to about 90%, about 30% to about 90%, about 35% to about 90%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, about 85% to about 90%, about 10% to about 85%, about 15% to about 85%, about 20% to about 85%, about 20% to about 80%, about 25% to about 80%, about 30% to about 80%, about 35% to about 80%, about 40% to about 80%, about 45% to about 80%, about 50% to about 80%, about 55% to about 80%, about 10% to about 75%, about 15% to about 75%, about 20% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 10% to about 50%, about 15% to about 50%, about 20% to about 50%, about 25% to about 50%, about 30% to about 50%, about 35% to about 50%, about 40% to about 50%, about 45% to about 50%, about 10% to about 45%, about 15% to about 45%, about 20% to about 45%, about 25% to about 45%, about 30% to about 45%, about 35% to about 45%, about 40% to about 45%, about 10% to about 40%, about 15% to about 40%, about 20% to about 40%, about 25% to about 40%, about 30% to about 40%, about 35% to about 40%, about 10% to about 35%, about 15% to about 35%, about 20% to about 35%, about 25% to about 35%, about 30% to about 35%, about 10% to about 30%, about 15% to about 30%, about 20% to about 30%, about 25% to about 30%, about 10% to about 25%, about 15% to about 25%, about 20% to about 25%, about 10% to about 20%, about 15% to about 20%, about 10% to about 15%, about 1%, about 10%, about 15%, about 20%, about 25%, about 30%, about 40%, about 50%, about 60%, about 70%, about 75%, about 80%, about 90%, about 95%, about 99%, or any range or value therebetween by weight of the total weight of the oil.

In any of the foregoing embodiments, the composition includes oil that includes free fatty acids derived from the second oil source, and optionally, a third oil source, and the free fatty acids of each of the second oil source and the third oil source can include two or more different fatty acids. By way of example, but not limitation, the free fatty acids derived from each of the second oil source and the optional third oil source can include two, three, four, five or more different fatty acids.

In any of the foregoing embodiments, the free fatty acids, including the two or more different fatty acids derived from the second oil source, can be, by way of example but not limitation, selected from the group consisting of caproic acid, enanthic acid, caprylic acid, pelargonic acid, capric acid, undecylic acid, lauric acid, tridecylic acid, myristic acid, pentadecylic acid, palmitic acid, palmitoleic acid, margaric acid, heptadecenoic acid, stearic acid, nonadecylic acid, vaccenic acid, oleic acid, linoleic acid, linolenic acid, stearidonic acid, octadecatetraenoic acid, arachidic acid, heneicosylic acid, gondoic acid, eicosadienoic acid, paullinic acid, behenic acid, tricosylic acid, erucic acid, lignoceric acid, docosahexaenoic acid, eicosapentaenoic acid, nervonic acid, and isomers thereof. Alternatively, the fatty acids derived from the second oil source, including the two or more different fatty acids can each independently be selected from the group consisting of a C6 fatty acid such as, by way of example but not limitation, C6:0, a C7 fatty acid, a C8 fatty acid such as, by way of example but not limitation, C8:0 fatty acid, a C9 fatty acid, a C10 fatty acid, a C11 fatty acid, a C12 fatty acid, a C13 fatty acid, a C14 fatty acid, a C15 fatty acid, a C16 fatty acid, a C17 fatty acid, a C18 fatty acid such as, by way of example, but not limitation, a C18:0 fatty acid, a C18:0 fatty acid, a C18:1 fatty acid, a C18:2 fatty acid, or a C18:3 fatty acid, a C19 fatty acid, a C20 fatty acid such as, by way of example, but not limitation, C20:5 fatty acid, a C21 fatty acid, a C22 fatty acid such as, by way of example, but not limitation, C22:6 fatty acid, a C23 fatty acid, and a C24 fatty acid. As used herein, C18 should be understood to refer to stearic acid (C18:0).

In any of the foregoing embodiments, the free fatty acids derived from the second oil source can have a fatty acid profile comprising two, three, four, five, six, seven, eight, nine, ten, eleven, or twelve of C6:0, C8:0, C10, C12, C14, C16, C18, C18:1, C18:2, C18:3, C20 and C22, wherein a range of each fatty is acid is between about 0.1% and 85% out of the total fatty acid content. By way of example, but not limitation, for each fatty acid, the amount can be from about 0.1% to about 85%, about 0.5% to about 85%, about 1% to about 85%, about 2% to about 85%, about 3% to about 85%, about 4% to about 85%, about 5% to about 85%, about 10% to about 85%, about 15% to about 85%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 0.1% to about 80%, about 0.5% to about 80%, about 1% to about 80%, about 2% to about 80%, about 3% to about 80%, about 4% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 0.1% to about 75%, about 0.5% to about 75%, about 1% to about 75%, about 2% to about 75%, about 3% to about 75%, about 4% to about 75%, about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 0.1% to about 70%, about 0.5% to about 70%, about 1% to about 70%, about 2% to about 70%, about 3% to about 70%, about 4% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 0.1% to about 65%, about 0.5% to about 65%, about 1% to about 65%, about 2% to about 65%, about 3% to about 65%, about 4% to about 65%, about 5% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 0.1% to about 60%, about 0.5% to about 60%, about 1% to about 60%, about 2% to about 60%, about 3% to about 60%, about 4% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 0.1% to about 55%, about 0.5% to about 55%, about 1% to about 55%, about 2% to about 55%, about 3% to about 55%, about 4% to about 55%, about 5% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 0.1% to about 50%, about 0.5% to about 50%, about 1% to about 50%, about 2% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 45%, about 0.5% to about 45%, about 1% to about 45%, about 2% to about 45%, about 3% to about 45%, about 4% to about 45%, about 5% to about 45%, about 10% to about 45%, about 15% to about 45%, about 20% to about 45%, about 25% to about 45%, about 30% to about 45%, about 35% to about 45%, about 40% to about 45%, about 0.1% to about 40%, about 0.5% to about 40%, about 1% to about 40%, about 2% to about 40%, about 3% to about 40%, about 4% to about 40%, about 5% to about 40%, about 10% to about 40%, about 15% to about 40%, about 20% to about 40%, about 25% to about 40%, about 30% to about 40%, about 35% to about 40%, about 0.1% to about 35%, about 0.5% to about 35%, about 1% to about 35%, about 2% to about 35%, about 3% to about 35%, about 4% to about 35%, about 5% to about 35%, about 10% to about 35%, about 15% to about 35%, about 20% to about 35%, about 25% to about 35%, about 30% to about 35%, about 0.1% to about 30%, about 0.5% to about 30%, about 1% to about 30%, about 2% to about 30%, about 3% to about 30%, about 4% to about 30%, about 5% to about 30%, about 10% to about 30%, about 15% to about 30%, about 20% to about 30%, about 25% to about 30%, about 0.1% to about 25%, about 0.5% to about 25%, about 1% to about 25%, about 2% to about 25%, about 3% to about 25%, about 4% to about 25%, about 5% to about 25%, about 10% to about 25%, about 15% to about 25%, about 20% to about 25%, about 0.1% to about 20%, about 0.5% to about 20%, about 1% to about 20%, about 2% to about 20%, about 3% to about 20%, about 4% to about 20%, about 5% to about 20%, about 10% to about 20%, about 15% to about 20%, about 0.1% to about 15%, about 0.5% to about 15%, about 1% to about 15%, about 2% to about 15%, about 3% to about 15%, about 4% to about 15%, about 5% to about 15%, about 10% to about 15%, about 0.1% to about 10%, about 0.5% to about 10%, about 1% to about 10%, about 2% to about 10%, about 3% to about 10%, about 4% to about 10%, about 5% to about 10%, about 0.1% to about 5%, about 0.5% to about 5%, about 1% to about 5%, about 2% to about 5%, about 3% to about 5%, about 4% to about 5%, about 0.1% to about 4%, about 0.5% to about 4%, about 1% to about 4%, about 2% to about 4%, about 3% to about 4%, about 0.1% to about 3%, about 0.5% to about 3%, about 1% to about 3%, about 2% to about 3%, about 0.1% to about 2%, about 0.5% to about 2%, about 1% to about 2%, about 0.1% to about 1%, about 0.5% to about 1%, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80% or 85% out of the total fatty acid content.

By way of example, but not limitation, where the FFA derived from the second oil source is derived from flaxseed oil, the two or more different fatty acids can comprise two or more fatty acids from C18:1 in an amount from about 10% to about 20%, C18:2 in an amount from about 10% to about 20%, and C18:3 in an amount from about 50% to about 65% out of the total fatty acid content of the FFA oil.

By way of example, but not limitation, where the second oil source is derived from flaxseed oil, the two or more different fatty acids can comprise two or more fatty acids from:
C18:3 in an amount from about 50% to about 70% out of the total fatty acid content, preferably from about 55% to about 65%, more preferably about 59.7%,
C18:2 in an amount from about 5% to about 30% out of the total fatty acid content, preferably from about 10% to about 20%, more preferably about 16%,
C18:1 in an amount from about 5% to about 30% out of the total fatty acid content, preferably from about 10% to about 20%, more preferably about 15.4%,
C16 in an amount from about 0.1% to about 15% out of the total fatty acid content, preferably from about 0.1% to about 10%, more preferably about 5.2%,
C18 in an amount from about 0.1% to about 10% out of the total fatty acid content, preferably from about 0.1% to about 5%, more preferably about 3.3%, and
C20 in an amount from about 0.1% to about 10% out of the total fatty acid content, preferably from about 0.1% to about 5%, more preferably about 0.4%.
   By way of further example, but not limitation, where the second oil source is derived from flaxseed oil, the respective oil can include from about 50% to about 70% MAGs, about 5% to about 25% DAGs, less than 5% TAGs, and about 5% to about 30% FFAs, based on the total weight of the respective oil.

In any of the foregoing embodiments, the FFA derived from the second oil source can be derived from an oil source(s) selected from a plant, an animal, a fish, an algal oil, and combinations thereof. In any of the foregoing embodiments, by way of example but not limitation, the FFA derived from the second oil source can be derived from olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, carrot seed oil, coffee bean oil, wheat germ oil, raspberry seed oil, jojoba oil, shea butter, edible oils, medicinal oils, and combinations thereof. In any of the foregoing embodiments, the FFA derived from the second oil source is derived from flaxseed oil.

In any of the foregoing embodiments, the composition includes oil that includes a mixture of monoacylglycerides, diacylglycerides, and free fatty acids derived from a third oil source, and the third oil source comprises a fatty acid profile that is different from each of the fatty acid profiles of the first oil source and second oil source. The third oil source can include two or more different fatty acids. By way of example, but not limitation, the free fatty acids derived from each of the third oil source can include two, three, four, five or more different fatty acids.

The mixture of monoacylglycerides, diacylglycerides, and free fatty acids derived from the third oil source are about 1% to about 80% by weight of the total weight of the oil. By way of example, but not limitation, the mixture of monoacylglycerides, diacylglycerides, and free fatty acids derived from the third oil source are at least 1%, at least 2%, at least 3%, at least 4%, at least 5%, at least 6%, at least 7%, at least 8%, at least 9%, least 10%, at least 15%, at least 20%, at least 25%, at least 30%, at least 35%, at least 40%, at least 45%, at least 50%, at least 55%, at least 60%, at least 65%, at least 70%, at least 75% by weight of the total weight of the oil. By way of further example, but not limitation, the oil can the free fatty acids, monoacylglycerides, and diacylglycerides in an amount of about 1% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 1% to about 75%, about 10% to about 75%, about 15% to about 75%, about 20% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 1% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 1% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 1% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 1% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 1% to about 50%, about 10% to about 50%, about 15% to about 50%, about 20% to about 50%, about 25% to about 50%, about 30% to about 50%, about 35% to about 50%, about 40% to about 50%, about 45% to about 50%, about 1% to about 45%, about 10% to about 45%, about 15% to about 45%, about 20% to about 45%, about 25% to about 45%, about 30% to about 45%, about 35% to about 45%, about 40% to about 45%, about 1% to about 40%, about 10% to about 40%, about 15% to about 40%, about 20% to about 40%, about 25% to about 40%, about 30% to about 40%, about 35% to about 40%, about 1% to about 35%, about 10% to about 35%, about 15% to about 35%, about 20% to about 35%, about 25% to about 35%, about 30% to about 35%, about 1% to about 30%, about 10% to about 30%, about 15% to about 30%, about 20% to about 30%, about 25% to about 30%, about 1% to about 25%, about 10% to about 25%, about 15% to about 25%, about 20% to about 25%, about 1% to about 20%, about 10% to about 20%, about 15% to about 20%, about 1% to about 15%, about 10% to about 15%, about 1% to about 10%, about 1%, about 10%, about 15%, about 20%, about 25%, about 30%, about 40%, about 50%, about 60%, about 70%, about 75%, about 80%, about 90%, about 95%, about 99%, or any range or value therebetween by weight of the total weight of the oil.

In any of the foregoing embodiments, the free fatty acids derived from the third oil source can include two or more different fatty acids. By way of example, but not limitation, the free fatty acids derived from the third oil source can include two, three, four, five or more different fatty acids.

In any of the foregoing embodiments, the free fatty acids, including the two or more different fatty acids derived from the third oil source, can be, by way of example but not limitation, selected from the group consisting of caproic acid, enanthic acid, caprylic acid, pelargonic acid, capric acid, undecylic acid, lauric acid, tridecylic acid, myristic acid, pentadecylic acid, palmitic acid, palmitoleic acid, margaric acid, heptadecenoic acid, stearic acid, nonadecylic acid, vaccenic acid, oleic acid, linoleic acid, linolenic acid, stearidonic acid, octadecatetraenoic acid, arachidic acid, heneicosylic acid, gondoic acid, eicosadienoic acid, paullinic acid, behenic acid, tricosylic acid, erucic acid, lignoceric acid, docosahexaenoic acid, eicosapentaenoic acid, nervonic acid, and isomers thereof. Alternatively, the fatty acids derived from the third oil source, including the two or more different fatty acids can each independently be selected from the group consisting of a C6 fatty acid such as, by way of example but not limitation, C6:0, a C7 fatty acid, a C8 fatty acid such as, by way of example but not limitation, C8:0 fatty acid, a C9 fatty acid, a C10 fatty acid, a C11 fatty acid, a C12 fatty acid, a C13 fatty acid, a C14 fatty acid, a C15 fatty acid, a C16 fatty acid, a C17 fatty acid, a C18 fatty acid such as, by way of example, but not limitation, a C18:0 fatty acid, a C18:0 fatty acid, a C18:1 fatty acid, a C18:2 fatty acid, or a C18:3 fatty acid, a C19 fatty acid, a C20 fatty acid such as, by way of example, but not limitation, C20:5 fatty acid, a C21 fatty acid, a C22 fatty acid such as, by way of example, but not limitation, C22:6 fatty acid, a C23 fatty acid, and a C24 fatty acid. As used herein, C18 should be understood to refer to stearic acid (C18:0).

In any of the foregoing embodiments, the free fatty acids derived from the third oil source can have a fatty acid profile comprising two, three, four, five, six, seven, eight, nine, ten, eleven, or twelve of C6:0, C8:0, C10, C12, C14, C16, C18, C18:1, C18:2, C18:3, C20 and C22, wherein a range of each fatty is acid is between about 0.1% and 85% out of the total fatty acid content. By way of example, but not limitation, for each fatty acid, the amount can be from about 0.1% to about 85%, about 0.5% to about 85%, about 1% to about 85%, about 2% to about 85%, about 3% to about 85%, about 4% to about 85%, about 5% to about 85%, about 10% to about 85%, about 15% to about 85%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 0.1% to about 80%, about 0.5% to about 80%, about 1% to about 80%, about 2% to about 80%, about 3% to about 80%, about 4% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 0.1% to about 75%, about 0.5% to about 75%, about 1% to about 75%, about 2% to about 75%, about 3% to about 75%, about 4% to about 75%, about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 0.1% to about 70%, about 0.5% to about 70%, about 1% to about 70%, about 2% to about 70%, about 3% to about 70%, about 4% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 0.1% to about 65%, about 0.5% to about 65%, about 1% to about 65%, about 2% to about 65%, about 3% to about 65%, about 4% to about 65%, about 5% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 0.1% to about 60%, about 0.5% to about 60%, about 1% to about 60%, about 2% to about 60%, about 3% to about 60%, about 4% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 0.1% to about 55%, about 0.5% to about 55%, about 1% to about 55%, about 2% to about 55%, about 3% to about 55%, about 4% to about 55%, about 5% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 0.1% to about 50%, about 0.5% to about 50%, about 1% to about 50%, about 2% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 45%, about 0.5% to about 45%, about 1% to about 45%, about 2% to about 45%, about 3% to about 45%, about 4% to about 45%, about 5% to about 45%, about 10% to about 45%, about 15% to about 45%, about 20% to about 45%, about 25% to about 45%, about 30% to about 45%, about 35% to about 45%, about 40% to about 45%, about 0.1% to about 40%, about 0.5% to about 40%, about 1% to about 40%, about 2% to about 40%, about 3% to about 40%, about 4% to about 40%, about 5% to about 40%, about 10% to about 40%, about 15% to about 40%, about 20% to about 40%, about 25% to about 40%, about 30% to about 40%, about 35% to about 40%, about 0.1% to about 35%, about 0.5% to about 35%, about 1% to about 35%, about 2% to about 35%, about 3% to about 35%, about 4% to about 35%, about 5% to about 35%, about 10% to about 35%, about 15% to about 35%, about 20% to about 35%, about 25% to about 35%, about 30% to about 35%, about 0.1% to about 30%, about 0.5% to about 30%, about 1% to about 30%, about 2% to about 30%, about 3% to about 30%, about 4% to about 30%, about 5% to about 30%, about 10% to about 30%, about 15% to about 30%, about 20% to about 30%, about 25% to about 30%, about 0.1% to about 25%, about 0.5% to about 25%, about 1% to about 25%, about 2% to about 25%, about 3% to about 25%, about 4% to about 25%, about 5% to about 25%, about 10% to about 25%, about 15% to about 25%, about 20% to about 25%, about 0.1% to about 20%, about 0.5% to about 20%, about 1% to about 20%, about 2% to about 20%, about 3% to about 20%, about 4% to about 20%, about 5% to about 20%, about 10% to about 20%, about 15% to about 20%, about 0.1% to about 15%, about 0.5% to about 15%, about 1% to about 15%, about 2% to about 15%, about 3% to about 15%, about 4% to about 15%, about 5% to about 15%, about 10% to about 15%, about 0.1% to about 10%, about 0.5% to about 10%, about 1% to about 10%, about 2% to about 10%, about 3% to about 10%, about 4% to about 10%, about 5% to about 10%, about 0.1% to about 5%, about 0.5% to about 5%, about 1% to about 5%, about 2% to about 5%, about 3% to about 5%, about 4% to about 5%, about 0.1% to about 4%, about 0.5% to about 4%, about 1% to about 4%, about 2% to about 4%, about 3% to about 4%, about 0.1% to about 3%, about 0.5% to about 3%, about 1% to about 3%, about 2% to about 3%, about 0.1% to about 2%, about 0.5% to about 2%, about 1% to about 2%, about 0.1% to about 1%, about 0.5% to about 1%, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80% or 85% out of the total fatty acid content.

By way of example, but not limitation, where the third oil source is oat oil, the two or more different fatty acids can comprise two or more fatty acids from:
C18:2 in an amount from about 40% to about 65% out of the total fatty acid content, preferably from about 45% to about 60%, more preferably about 53.8%,
C18:1 in an amount from about 10% to about 35% out of the total fatty acid content, preferably from about 15% to about 30%, more preferably about 22.5%,
C16 in an amount from about 0.1% to about 25% out of the total fatty acid content, preferably from about 5% to about 20%, more preferably about 11.4%,
C18:3 in an amount from about 0.1% to about 20% out of the total fatty acid content, preferably from about 5% to about 15%, more preferably about 7.2%,
C18 in an amount from about 0.1% to about 15% out of the total fatty acid content, preferably from about 0.1% to about 10%, more preferably about 4.3%, and
C20 in an amount from about 0.1% to about 10% out of the total fatty acid content, preferably from about 0.1% to about 5%, more preferably about 0.8%.
   By way of further example, but not limitation, where the third oil source is derived from oat oil, the respective oil can include from about 60% to about 95% MAGs, about 0.1% to about 50% DAGs, less than 5% TAGs, and about 5% to about 30% FFAs, based on the total weight of the EMO and/or FFA oil.

In any of the foregoing embodiments, the composition includes fatty acids derived from a third oil source, and the fatty acids are about 5% to about 30% by weight of the total weight of the mixture of monoacylglycerides, diacylglycerides, and free fatty acids derived from the third oil source. By way of example, but not limitation, the free fatty acids derived from the third oil source are at least 5%, at least 6%, at least 7%, at least 8%, at least 9%, least 10%, at least 15%, at least 20%, at least 25%, by weight of the total weight of the mixture of monoacylglycerides, diacylglyceride, and free fatty acids derived from the third oil source. By way of further example, but not limitation, the mixture of monoacylglycerides, diacylglyceride, and free fatty acids can include about 5% to about 30%, about 10% to about 30%, about 15% to about 30%, about 20% to about 30%, about 25% to about 30%, about 5% to about 25%, about 10% to about 25%, about 15% to about 25%, about 20% to about 25%, about 5% to about 20%, about 10% to about 20%, about 15% to about 20%, about 5% to about 15%, about 10% to about 15%, about 5% to about 10%, about 5%, about 10%, about 15%, about 20%, about 25%, about 30%, or any range or value therebetween by weight of the total weight of the mixture of monoacylglycerides, diacylglyceride, and free fatty acids derived from the third oil source.

In any of the foregoing embodiments, the composition includes monoacylglycerides derived from a third oil source, and the monoacylglycerides are about 60% to about 95% by weight of the total weight of the mixture of monoacylglycerides and free fatty acids derived from the third oil source. By way of example, but not limitation, the monoacylglycerides derived from the third oil source are at least 60%, 65%, 70%, 75%, 80%, 85%, 90%, or 95% by weight of the total weight of the mixture of monoacylglycerides, diacylglyceride, and free fatty acids derived from the third oil source. By way of further example, but not limitation, the mixture of monoacylglycerides, diacylglyceride, and free fatty acids derived from the third oil source can include about 60% to about 95%, about 65% to about 95%, about 70% to about 95%, about 75% to about 95%, about 75% to about 95%, about 80% to about 95%, about 85% to about 95%, about 90% to about 95%, about 60% to about 90%, about 65% to about 90%, about 70% to about 90°/a, about 75% to about 90%, about 80% to about 90%, about 85% to about 90%, about 90% to about 95%, about 60% to about 85%, about 65% to about 85%, about 70% to about 85%, about 75% to about 85%, about 80% to about 85%, about 60% to about 80%, about 65% to about 80%, about 70% to about 80%, about 75% to about 80%, about 80% to about 85%, about 60% to about 75%, about 65% to about 75%, about 70% to about 75%, about 60% to about 70%, about 65% to about 70%, about 60% to about 65%, about 60%, about 65%, about 70%, about 75%, about 80%, about 85%, about 90% about 95%, or any range or value therebetween by weight of the total weight of the mixture of monoacylglycerides, diacylglyceride, and free fatty acids derived from the third oil source.

In any of the foregoing embodiments, the composition includes diacylglycerides derived from a third oil source, and the diacylglycerides are about 1% to about 20% by weight of the total weight of the mixture of monoacylglycerides, diacylglycerides, and free fatty acids derived from the third oil source. By way of example, but not limitation, the diacylglycerides derived from the third oil source are at least 1%, 2%, 3%, 4%, 5%, 6%, 7%, 8%, 9%, 10%, 11%, 12%, 13%, 14%, 15%, 16%, 17%, 18%, 19%, or 20% by weight of the total weight of the mixture of monoacylglycerides, diacylglyceride, and free fatty acids derived from the third oil source. By way of further example, but not limitation, the mixture of monoacylglycerides, diacylglyceride, and free fatty acids derived from the third oil source can include about 1% to about 20%, about 2% to about 20%, about 4% to about 20%, about 6% to about 20%, about 8% to about 20%, about 10% to about 20%, about 12% to about 20%, about 14% to about 20%, about 16% to about 20%, about 18% to about 20%, 2% to about 18%, about 4% to about 18%, about 6% to about 18%, about 8% to about 18%, about 10% to about 20%, about 12% to about 18%, about 14% to about 18%, about 16% to about 18%, 4% to about 16%, about 6% to about 18%, about 8% to about 16%, about 10% to about 16%, about 12% to about 16%, about 14% to about 16%, about 6% to about 14%, about 8% to about 14%, about 10% to about 14%, about 12% to about 14%, about 8% to about 12%, about 10% to about 12%, about 3%, about 5%, about 7%, about 9%, about 11%, about 13%, about 15%, about 17% about 19% or any range or value therebetween by weight of the total weight of the mixture of monoacylglycerides, diacylglyceride, and free fatty acids derived from the third oil source.

In any of the foregoing embodiments, the composition and/or oil can include 80% or less, 70% or less, 60% or less, 50% or less, 40% or less, 30% or less, 25% or less, 20% or less, 15% or less, 10% or less, 5% or less, 4% or less, 3% or less, 2% or less, 1% or less, or is substantially free of triacylglycerols (TAGs).

By way of further example, the composition and/or oil can have no measurable TAGs as assessed qualitatively by Thin Layer Chromatography (TLC). In this method, components of the oil are analyzed using TLC plates (Analtech Uniplate Silica Gel GHL with inorganic binder, 20×20 cm, 250 micrometers), using a solvent (Hexane: Diethyl Ether: Acetic Acid (70:30:1) solution. Typical sample sizes are 0.2 microliters. After the solvent front runs to near the top of the plate (about 1 cm), plates are removed from the TLC tank and the solvent evaporated in a fume hood. The components are visualized with iodine vapors (at room temperature) in a TLC tank and relative intensities estimated by colorimetric imaging.

Components can also be measure quantitatively by standard HPLC and GC/MS methods. By way of still further example, the composition and/or the oil can have no measurable TAGs as assessed by gas chromatography (GC) or high performance liquid chromatography (HPLC). By way of example, but not limitation, HPLC can be performed under the following HPLC conditions: C18 column, 4.6 mm I.D.×250 mm, 5 micrometers; column temp at 30° C.; charged aerosol detector, mobile phase A consisting of 75% acetonitrile, 12% methanol, 8% water, 4% tetrahydrofuran, and 0.3% acetic acid; mobile phase B consisting of 90% acetone and 10% acetonitrile; a gradient profile of: 100% A at 1.5 m/min from 0-5 min, 100% A to 96% A at 1.5 m/min from 5 to 11 min, 96% A to 37% A at 1.5 m/min from 11 to 24 min, 37% A to 32% A at 1.5 m/min from 24 to 35 min, 32% A to 18% A at 1.5 m/min from 35 to 45 min, 18% A to 15% A at 1.9 mL/min from 45 to 58 min, 15% A to 5% A at 1.9 m/min from 58 to 60 min, 5% A to 100% A at 1.5 m/min from 60 to 63 min, 100% A at 1.5 mL/min from 63 to 65 min, using MAG, DAG, TAG and FFA calibration standards. Methods of quantitative measurements of glycerides and free fatty acids are described, by way of example but not limitation, Simultaneous Analysis of Glycerides (Mono-, Di-, and Triglycerides) and Free Fatty Acids in Palm Oil, Application Note 1039, Thermo Fisher Scientific, Inc., 2012, Annex 5.

In any of the foregoing embodiments, the free fatty acids, monoacylglycerides, and diacylglycerides derived from the third oil source can be derived, from an oil source(s) selected from a plant, an animal, a fish, an algal oil, and combinations thereof. In any of the foregoing embodiments, by way of example but not limitation, the free fatty acids, monoacylglycerides, and diacylglycerides, derived from the third oil source can be derived from olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, carrot seed oil, coffee bean oil, wheat germ oil, raspberry seed oil, jojoba oil, shea butter, edible oils, medicinal oils, and combinations thereof. By way of example but not limitation, the free fatty acids, monoacylglycerides, and diacylglycerides derived from the third oil source are derived from oat oil. By way of further example but not limitation, the free fatty acids, monoacylglycerides, and diacylglycerides derived from the third oil source are derived from an enzyme modified oil (EMO).

In any of the foregoing embodiments, the composition, the free fatty acids derived from the first oil source, the free fatty acids derived from the second oil source, and/or the free fatty acids, monoacyglycerides, and diacylglycerides derived from the third oil source can have a triacylglycerol (TAG) content of 5% or less TAGs by weight based on the total weight of the composition and/or oil, respectively. By way of example, but not limitation, the TAG content of any of the foregoing can be equal to or less than 4%, equal to or less than 3%, equal to or less than 2%, equal to or less than 1% by weight based on the total weight of the composition and/or the respective oil source, or the composition and/or the respective oil can be substantially free of TAGs.

In any of the foregoing embodiments, the composition, the free fatty acids derived from the first oil source, the free fatty acids derived from the second oil source, and/or the free fatty acids, monoacylglycerides, and diacylglycerides derived from the third oil source can further include non-oil ingredients derived from and naturally present in the oil source(s) from which the oils are derived, respectively. By way of example, but not limitation, the non-oil ingredients derived from and naturally present in the first oil source, second oil source, and/or third oil source can include glycosphingolipids such as ceramide phosphates, glycoglycerolipids such as monoglactosyl diacylglycerols, phosphatidyl alcohols such as phosphatidyl methanol, steroids such as sitosteryl esters, natural lipids such as campesterol esters, sphingolipids, phosphatidyl glycerol, wax esters, sphingomyelin, phosphatides, phytosterols (campesterol, stigmasterol, sitosterol), cholesterol, tocopherols, tocotrienols, carotenes, xanthophylls, betaxanthins, chlorophyll, long chain alcohols, polyphenols, terpenes (cycloartanol, cycloarterenol, 24-methylene cycloarterenol), terpenoids (squalene), tocopherol, vitamins, paraffins, isothiocyanates, oxazolidinthiones, glucosinolates, sanguinarine, gossypols, glycosylated alkaloids, phospholipids, sphingosine, diacylglyceride ethers, glucolipids, phytic acid, quinic acid, oxalic acid, tartaric acid, anacardic acid, malic acid, caftaric acid, coutaric acid, fertaric acid, phenylethanoids, hydroxycinnamic acids, phenolic acids, phytoestrogens, lignans, isoflavonoids, flavonoids, monoterpenes, diterpenes, triterpenoids, coenzyme, or combinations thereof. By way of further example, but not limitation, the non-oil ingredients derived from and naturally present in the first oil source, second oil source, or third oil source an include ceramide phosphate, monoglactodiacylglycerol, phosphatidylmethanol, sitosteryl ester, campesterol ester, sphingolipids, phosphatidyl glycerol, wax esters, sphingomyelin, or combinations thereof. By way of still further example, but not limitation, the non-oil ingredients derived from and naturally present in the first oil source, second oil source, or third oil source can include α-tocopherol, ß-tocopherol, δ-tocopherol, γ-tocopherol, α-tocotrienol, ß-tocotrienol, δ-tocotrienol, γ-tocotrienol, or combinations thereof. By way of example, but not limitation, the non-oil components can be determined by LC/MS/MS analysis.

In any of the foregoing embodiments, the composition and/or the oil can include a processed oil from an oil source that has been subjected to an enzymatic treatment selected from the group consisting of enzymatic hydrolysis to yield free fatty acids and glycerol with removal of glycerol to yield free fatty acid oil, enzymatic re-esterification of free fatty acids (FFA) to yield monoacylglycerols (MAGs) and free fatty acids and, optionally, diacylglycerols (DAGs), and both enzymatic hydrolysis to yield free fatty acids and glycerol with removal of glycerol to yield FFA oil and enzymatic re-esterification of FFA to yield monoacylglycerols (MAGs) and free fatty acids and, optionally, diacylglycerols (DAGs) to yield EMO. By way of example, but not limitation, the FFA derived from the first oil source, the FFA derived from the second oil source, and/or the FFA, MAG, and DAG derived from the third oil source or a portion thereof can be an enzyme-modified oil (EMO) that has been subjected to enzymatic hydrolysis to yield free fatty acids and glycerol, separation of the glycerol from the free fatty acid oil, and subsequent enzymatic re-esterification to yield monoacylglycerols, free fatty acids and, optionally, diacylglcyerols.

In any of the foregoing embodiments, the MAGs can include two or more different MAGs. By way of example, but not limitation, the MAGs can include two, three, four, five or more different MAGs. By way of further example, but not limitation, the MAGs can be selected from the group consisting of monocaproin, monocaprylin, monocaprin, monolaurin, monomyristin, monopalmitin, monostearin, monoolein, monolinolein, monolinolenin, monoeicosapentaenoin, monodocosahexaenoin, and combinations thereof.

It should be understood, in any of the foregoing embodiments, that the first oil source, the second oil source, and the third oil source can be the same type of oil or different types of oil and that each of the first oil source, the second oil source, and the third oil source can be a single type of oil (e.g. coconut oil, oat oil, or flaxseed oil) or a combination of oils as described further herein. It should likewise be further understood that the respective oils can be the result of a blend of oils, respectively, which have been processed independently and later combined such that the first oil source, the second oil source, and the third oil source are each the combination of oil sources used to produce the different respective oils that have been combined.

In any of the foregoing embodiments, the second oil source and/or the third oil source can include a fatty acid profile that is within about 10% of the fatty acid profile from the oil source used to yield the oil for at least one, two or more fatty acids. In aspects where more than one oil source is used to form the oil, the fatty acid profile can be within about 10% of the fatty acid profile from the oil sources according to the respective amounts of each oil source used to form the oil for at least one, two or more fatty acids. By way of example, but not limitation, the fatty acid profile of the second oil source and/or the third oil source for one, two, three, four, five, six, seven, eight, nine, ten or more fatty acids can be within 10% of the fatty acid profile for the same fatty acids in the oil source(s), according to the respective amounts of each oil source used to form the oil if two or more oil sources are used, or for the oil source where only one oil source is used to form the oil. By way of further example, but not limitation, the fatty acid profile can be within about 10%, 9%, 8%, 7%, 6%, 5%, 4%, 3%, 2%, or 1% for each fatty acid in the oil as compared to the oil source(s). For example, if the second oil source has 15% C18 fatty acid, the EMO can have between about 5-25%, 6-24%, 7-23%, 8-22%, 9-21%, 10-20%, 11-19%, 12-18%, 13-17%, 14-16% or about 15% C18 fatty acid. Exemplary fatty acid profiles are provided in the Tables below which also include exemplary EMO compositions based on experimental data. It should be understood that for fatty acid profiles, amounts of fatty acid can naturally vary by about 3-5% depending on species, growing conditions and other variables. It should be understood that the fatty acid profile that is expected can be estimated based on known fatty acid profiles and the amount of EMO and/or FFA oil.

TABLE B
Fatty acid Profiles and FFA, MAG, DAG,
and TAG content of various EMOs

| Fatty | Flaxseed | MCT | Rosehip | Sesame | Hempseed | Coconut |
|---|---|---|---|---|---|---|
| Acid | Oil | Oil | Oil | Oil | Oil | Oil* |
| C6:0 | 0.0 | 0.06 | 0.0 | 0.0 | 0.0 | 0.0 |
| C8:0 | 0.0 | 57.42 | 0.0 | 0.0 | 0.0 | 0.0 |
| C10 | 0.0 | 41.96 | 0.0 | 0.0 | 0.0 | 6.0 |
| C12 | 0.0 | 0.32 | 0.0 | 0.0 | 0.0 | 47.0 |
| C14 | 0.0 | 0.0 | 0.0 | 0.0 | 0.0 | 18.0 |
| C16 | 5.2 | 0.0 | 4.5 | 7.7 | 6.6 | 9.0 |
| C18 | 3.3 | 0.0 | 2.2 | 14.3 | 2.6 | 3.0 |
| C18:1 | 15.4 | 0.0 | 19.9 | 40.1 | 11.5 | 2.0 |
| C18:2 | 16.0 | 0.0 | 50.8 | 37.7 | 58.6 | 2.0 |
| C18:3 | 59.7 | 0.0 | 20.7 | 0.2 | 19.2 | 0.0 |
| C20 | 0.4 | 0.0 | 1.9 | 0.0 | 1.5 | 0.0 |
| C22 | 0.0 | 0.0 | 0.0 | 0.0 | 0.0 | 0.0 |
| EMO | | | | | | |
| MAG | 57.9 | est. 64 | 54.6 | 62.5 | 69.9 | est. 72 |
| DAG | 16.5 | est. 15 | 16.5 | 17.9 | 11 | est. 16 |
| TAG | BLD | BLD | BLD | BLD | BLD | BLD |
| FFA | 18 | 21 | 16 | 12 | 9 | 12 |

TABLE C
Fatty acid profiles and FFA, MAG, DAG, and TAG content
of fish, algae, oat, canola, olive, and almond oils

| Fatty | Fish | Algae | Oat | Canola | Olive | Almond |
|---|---|---|---|---|---|---|
| Acid | Oil* | Oil* | Oil | Oil | Oil* | Oil |
| C6:0 | 0.0 | 0.0 | 0.0 | 0.0 | 0.0 | 0.0 |
| C8:0 | 0.0 | 0.0 | 0.0 | 0.0 | 0.0 | 0.0 |
| C10 | 0.0 | 0.0 | 0.0 | 0.0 | 0.0 | 0.0 |
| C12 | 0.0 | 0.0 | 0.0 | 0.0 | 0.0 | 0.0 |
| C14 | 9.4 | 1.0 | 0.0 | 0.0 | 0.0 | 0.0 |
| C16 | 26.9 | 18.5 | 11.4 | 4.6 | 13.0 | 7.0 |
| C18 | 0.0 | 1.9 | 4.3 | 1.8 | 3.0 | 2.0 |
| C18:1 | 21.8 | 30.6 | 22.5 | 62.8 | 71.0 | 69.0 |
| C18:2 | 2.5 | 22.8 | 53.8 | 21.8 | 10.0 | 17.0 |
| C18:3 | 1.7 | 16.5 | 7.2 | 6.5 | 1.0 | 0.0 |
| C20 | 14.5 | 1.5 | 0.8 | 2.6 | 0.0 | 0.0 |
| C22 | 23.2 | 0.5 | 0.0 | 0.0 | 0.0 | 0.0 |
| EMO | | | | | | |
| MAG | est. 56 | | 65.9 | 60.9 | 55.9 | 74.9 |
| DAG | est. 14 | | 9.4 | 16.3 | 14.4 | 12.5 |
| TAG | 16 | | BLD | BLD | BLD | BLD |
| FFA | 12 | | 13 | 13.9 | 20 | 10 |

TABLE D
Fatty acid profiles and FFA, MAG, DAG, and TAG content of cottonseed,
peanut, sunflower, corn, safflower, and soybean oils

| Fatty | Cottonseed | Peanut | Sunflower | Corn | Safflower | Soybean |
|---|---|---|---|---|---|---|
| Acid | Oil* | Oil* | Oil* | Oil* | Oil* | Oil* |
| C6:0 | 0.0 | 0.0 | 0.0 | 0.0 | 0.0 | 0.0 |
| C8:0 | 0.0 | 0.0 | 0.0 | 0.0 | 0.0 | 0.0 |
| C10 | 0.0 | 0.0 | 0.0 | 0.0 | 0.0 | 0.0 |
| C12 | 0.0 | 0.0 | 0.0 | 0.0 | 0.0 | 0.0 |
| C14 | 1.0 | 0.0 | 0.0 | 0.0 | 0.0 | 0.0 |
| C16 | 22.0 | 11.0 | 7.0 | 11.0 | 7.0 | 11.0 |
| C18 | 3.0 | 2.0 | 5.0 | 2.0 | 2.0 | 4.0 |
| C18:1 | 19.0 | 48.0 | 19.0 | 28.0 | 13.0 | 24.0 |
| C18:2 | 54.0 | 32.0 | 68.0 | 58.0 | 78.0 | 54.0 |
| C18:3 | 1.0 | 0.0 | 1.0 | 1.0 | 0.0 | 7.0 |
| C20 | 0.0 | 0.0 | 0.0 | 0.0 | 0.0 | 0.0 |
| C22 | 0.0 | 0.0 | 0.0 | 0.0 | 0.0 | 0.0 |
| EMO | | | | | | |
| MAG | | | 58.3 | | | |
| DAG | | | 25.5 | | | |
| TAG | | | BLD | | | |
| FFA | | | 9 | | | |

TABLE E
Fatty acid profiles of cacao, grapeseed oil, palm, olein,
palm kernel, shea, walnut, and macadamia nut oils

| | | | | | Palm | | | |
|---|---|---|---|---|---|---|---|---|
| Fatty | Cacao | Grapeseed | Palm | Palm | Kernel | Shea | Walnut | Macadamia |
| Acid | Oil* | Oil* | Oil* | Olein* | Oil* | Oil* | Oil* | Nut Oil |
| C6:0 | 0.0 | 0.0 | 0.0 | 0.0 | 0.0 | 0.0 | 0.0 | 0.0 |
| C8:0 | 0.0 | 0.0 | 0.0 | 0.0 | 0.0 | 0.0 | 0.0 | 0.0 |
| C10 | 0.0 | 0.0 | 0.0 | 0.0 | 4.0 | 0.0 | 0.0 | 0.0 |
| C12 | 0.0 | 0.0 | 0.0 | 0.0 | 48.0 | 1.0 | 0.0 | 0.0 |
| C14 | 0.0 | 0.0 | 1.0 | 1.0 | 16.0 | 0.0 | 0.0 | 1.2 |
| C16 | 25.0 | 8.0 | 45.0 | 37.0 | 8.0 | 4.0 | 11.0 | 41 |
| C18 | 38.0 | 4.0 | 4.0 | 4.0 | 3.0 | 39.0 | 5.0 | 5.4 |
| C18:1 | 32.0 | 15.0 | 40.0 | 46.0 | 15.0 | 44.0 | 28.0 | 41 |
| C18:2 | 3.0 | 73.0 | 10.0 | 11.0 | 2.0 | 5.0 | 51.0 | 2.7 |
| C18:3 | 0.0 | 0.0 | 0.0 | 0.0 | 0.0 | 0.0 | 5.0 | 1 |
| C20 | 0.0 | 0.0 | 0.0 | 0.0 | 0.0 | 0.0 | 0.0 | 6.5 |
| C22 | 0.0 | 0.0 | 0.0 | 0.0 | 0.0 | 0.0 | 0.0 | 1.1 |

TABLE F
Fatty acid profiles and FFA, MAG, DAG, and TAG
content of pomegranate and shea butter oils

| | Fatty | | Fatty | Shea |
|---|---|---|---|---|
| | Acid | Pomegranate* | Acid | butter* |
| | C6:0 | 0.0 | C6:0 | |
| | C8:0 | 0.0 | C8:0 | |
| | C10 | 0.0 | C10 | |
| | C12 | 0.5 | C12 | |
| | C14 | 0.5 | C14 | |
| | C16 | 24.6 | C16 | 4.0 |
| | C18 | 8.9 | C18 | 41.5 |
| | C18:1 | 21.6 | C18:1 | 46.4 |
| | C18:2 | 28.9 | C18:2 | 6.6 |
| | C18:3 | 3.8 | C18:3 | |
| | C20 | 0.9 | C20 | 1.3 |
| | C22 | 1.3 | C22 | |
| EMO | | | | |
| | MAG | 54.0 | | |
| | DAG | 14.1 | | |
| | TAG | 2.7 | | |
| | FFA | 15 | | |

TABLE G
Fatty acid profiles of carrot seed, coffee bean,
avocado, wheat germ, raspberry, and jojoba oils

| Fatty | Carrot | Coffee | Avocado | Wheat | Raspberry | Jojoba |
|---|---|---|---|---|---|---|
| Acid | seed oil* | bean oil* | oil* | germ oil* | seed oil* | oil* |
| C6:0 | | | | | | |
| C8:0 | | | | | | |
| C10 | | | | | | |
| C12 | | | | | | |
| C14 | | | 0.3 | | | |
| C16 | 10.0 | 46.1 | 23.6 | 16 | 2.7 | 1.6 |
| C18 | 2.1 | 6.6 | | 0.8 | 1.0 | 0.1 |
| C18:1 | 59.4 | 8.0 | 47.2 | 13 | 12.0 | 11.2 |
| C18:2 | 11.8 | 32.9 | 13.4 | 56 | 54.5 | |
| C18:3 | | | 1.6 | 8 | 29.1 | |
| C20 | | 1.9 | 1.3 | | | 70.9 |
| C22 | | | 8.9 | | | 14.4 |

*Fatty acid profile values derived from literature;
BLD = below limit of detection

In any of the foregoing embodiments, the compositions include an antimicrobial composition of oil that include a fast-acting FFA oil component derived from the first oil source including capric acid, caprylic acid, or a combination thereof. Such antimicrobial compositions with medium chain fatty acids with carbon lengths of 8 and 10 interfere with microorganism cellular metabolism and can kill the microorganisms quickly or on contact. Such fast-acting compositions are generally non-specific. In any of the foregoing embodiments, the compositions include an antimicrobial composition of oil that includes a fast-acting FFA derived from the first oil source including capric acid, caprylic acid, or a combination thereof, and a slow-acting oil component that includes free fatty acids derived from a second oil source and/or a third oil source, which are anti-microbial by slowly interacting with cellular membranes of microorganisms to disrupt such membrane integrity. By way of example but not limitation, slow-acting oil component can include long-chain, polyunsaturated MAGs and FFA blends, in some embodiments.

### Organic Acids

In any of the foregoing embodiments, the composition can further include an organic acid, an organic acid salt, or a combination thereof. By way of example but not limitation, the composition includes an organic acid that is lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, salicylic acid, or any combination thereof. By way of example but not limitation, the organic acid salt is a salt of lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof. By way of example but not limitation, the organic acid salt is a sodium salt or a potassium salt of benzoic acid.

In any of the foregoing embodiments, the composition can further include an organic acid, an organic acid salt, or a combination thereof, where the organic acid, organic acid salt, or combination thereof is about 0.001 to about 10%, about 0.01 to about 10%, about 0.1% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0.001 to about 9%, about 0.01 to about 9%, about 0.1% to about 9%, about 1% to about 9%, about 2% to about 9%, about 3% to about 9%, about 4% to about 9%, about 5% to about 9%, about 6% to about 9%, about 7% to about 9%, about 8% to about 8%, about 0.001 to about 8%, about 0.01 to about 8%, about 0.1% to about 8%, about 1% to about 8%, about 2% to about 8%, about 3% to about 8%, about 4% to about 8%, about 5% to about 8%, about 6% to about 8%, about 7% to about 8%, about 0.001 to about 7%, about 0.01 to about 7%, about 0.1% to about 7%, about 1% to about 7%, about 2% to about 7%, about 3% to about 7%, about 4% to about 7%, about 5% to about 7%, about 6% to about 7%, about 0.001 to about 6%, about 0.01 to about 6%, about 0.1% to about 6%, about 1% to about 6%, about 2% to about 6%, about 3% to about 6%, about 4% to about 6%, about 5% to about 6%, about 0.001 to about 5%, about 0.01 to about 5%, about 0.1% to about 5%, about 1% to about 5%, about 2% to about 5%, about 3% to about 5%, about 4% to about 5%, about 0.001 to about 4%, about 0.01 to about 4%, about 0.1% to about 4%, about 1% to about 4%, about 2% to about 4%, about 3% to about 4%, about 0.001 to about 3%, about 0.01 to about 3%, about 0.1% to about 3%, about 1% to about 3%, about 2% to about 3%, about 0.001 to about 2%, about 0.01 to about 2%, about 0.1% to about 2%, about 1% to about 2%, about 0.001 to about 1%, about 0.01 to about 1%, about 0.1% to about 1%, about 0.001 to about 0.01%, or any range or value therebetween by weight of the total weight of composition.

In any of the foregoing embodiments, the composition can further include a surfactant and/or an emulsifier. By way of example but not limitation, the composition can include a surfactant that is an anionic surfactant, a cationic surfactant, an amphoteric surfactant, a zwitterionic surfactant, a nonionic surfactant, or any combination thereof.

By way of example but not limitation, the amphoteric surfactant is a derivative of an aliphatic secondary or tertiary amine in which the aliphatic radical can be a straight or branched chain, and wherein one of the aliphatic substituents contains from about 8 to about 18 carbon atoms and one contains an anionic water solubilizing group, e.g., carboxy, sulfonate, sulfate, phosphate, or phosphonate. Non-limiting examples of compounds falling within this definition are sodium 3-dodecyl-aminopropionate, cocamidopropyl betaine, sodium 3-dodecylaminopropane sulfonate, sodium lauryl sarcosinate, N-alkyltaurines, such as the one prepared by reacting dodecylamine with sodium isethionate, N-higher alkyl aspartic acids, or any combination thereof. Other non-limiting examples of amphoteric surfactants include amphoacetates and diamphoacetates, such as sodium lauroamphoacetate, sodium cocoamphoactetate, disodium lauroamphoacetate, disodium cocodiamphoacetate, or any combination thereof.

By way of example but not limitation, the nonionic surfactant is an alkyl glucoside, alkyl polyglucanoside, polyhydroxy fatty acid amide, alkoxylated fatty acid ester, lathering sucrose ester, amine oxide, C8-C14 glucose amide, C8-C14 alkyl polyglucoside, sucrose cocoate, sucrose laurate, glyceryl monohydroxystearate, steareth-2, propylene glycol stearate, PEG-2 stearate, sorbitan monostearate, glyceryl stearate, laureth-2, steareth-2, lauramine oxide, cocoamine oxide, or any combination thereof.

By way of example but not limitation, the cationic surfactant is stearyl trimethyl ammonium chloride, cetyl trimethyl ammonium chloride, behenyl trimethyl ammonium chloride, distearyl dimethyl ammonium chloride, an asymmetric dialkyl quaternized ammonium salt cationic surfactant, or any combination thereof.

By way of example but not limitation, the zwitterionic surfactant is zwitterionic surfactant includes betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, or any combination thereof. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines; and amidosulfobetaines, wherein the RCONH(CH₂)₃ radical is attached to the nitrogen atom of the betaine, or any combination thereof.

By way of example but not limitation, the anionic surfactant is ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

In one embodiment, the composition comprises sodium laureth sulfate and cocamidopropyl betaine, wherein the sodium laureth sulfate is present in an amount from about 5% to about 15%, from about 5% to about 10%, from about 6% to about 8%, from about 8% to about 12%, or at about 6%, at about 7%, at about 8%, at about 9%, at about 10%, at about 11%, at about 12%, at about 13%, or at about 14% by weight of the total weight of the composition, and the cosamidopropyl betain is from about 0.25% to about 5%, or about 0.5%, about 1.0%, about 1.5%, about 2%, about 2.5%, about 3%, about 3.5%, about 4%, about 4.5% or about 5% by weight of the total weigh of the composition.

In any of the foregoing embodiments, the surfactant and/or emulsifier is present in the composition in an amount of about 1% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 1% to about 45%, about 5% to about 45%, about 10% to about 45%, about 15% to about 45%, about 20% to about 45%, about 25% to about 45%, about 30% to about 45%, about 35% to about 45%, about 40% to about 45%, about 1% to about 40%, about 5% to about 40%, about 10% to about 40%, about 15% to about 40%, about 20% to about 40%, about 25% to about 40%, about 30% to about 40%, about 35% to about 40%, about 1% to about 35%, about 5% to about 35%, about 10% to about 35%, about 15% to about 35%, about 20% to about 35%, about 25% to about 35%, about 30% to about 35%, about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 7% to about 14%, about 7.5% to about 13.5%, about 10% to about 15%, about 1% to about 10%, about 5% to about 10%, about 1% to about 5%, about 1%, about 5%, about 10%, about 15%, about 20%, about 25%, about 30%, about 35%, about 40%, about 45%, about 50%, or any range or value therebetween by weight of the total weight of composition.

In any of the foregoing embodiments, the composition can further include an emulsifier. By way of example but not limitation, the emulsifier is polyoxyethylene oleyl ether, PEG-40 stearate, ceteareth-12, ceteareth-30, PEG-100 stearate, methyl myristate, isopropyl myristate, glyceryl stearate, steareth-2, steareth-20, dimethicone copolyol, Polysorbate 20 (Tween 20), Polysorbate 40 (Tween 40), Polysorbate 60 (Tween 60), Polysorbate 80 (Tween 80), lauramide DEA, cocamide DEA, cocamide MEA, phospholipid PTC, alginate, carrageenans, glucate DO, methylcellulose, polyvinyl alcohol, cocamidopropyl phosphatidyl PG-dimonium chloride, stearic acid, magnesium stearate, milk amino acids, triethanolamine, magnesium aluminum silicate, or any combination thereof.

In any of the foregoing embodiments, the composition can further include a chelating agent. By way of example but not limitation, the composition can include a chelating agent that is a tetrasubstituted sodium imino disuccinic acid, ethylene diamine tetraacetate (EDTA), a tetrasubstituted sodium salts of L-glutamic-N, N-diacetic acid, or any combination thereof.

In any of the foregoing embodiments, the composition can further include a chelating agent, where the chelating agent is in an amount of about 0.01% to about 1%, about 0.001% to about 0.9%, about 0.001% to about 0.8%, about 0.001% to about 0.7%, about 0.001% to about 0.6%, about 0.001% to about 0.5%, about 0.001% to about 0.4%, about 0.001% to about 0.3%, about 0.001% to about 0.2%, about 0.001% to about 0.1%, about 0.001% to about 0.01%, 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01% to about 0.1%, 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, 0.2% to about 0.9%, 0.2% about 0.8%, about 0.2% to about 0.7%, about 0.2% to about 0.6%, about 0.2% to about 0.5%, about 0.2% to about 0.4%, about 0.2% to about 0.3%, about 0.3% to about 1%, 0.3% to about 0.9%, 0.3% about 0.8%, about 0.3% to about 0.7%, about 0.3% to about 0.6%, about 0.2% to about 0.5%, about 0.3% to about 0.4%, about 0.4% to about 1%, 0.4% to about 0.9%, 0.4% about 0.8%, about 0.4% to about 0.7%, about 0.4% to about 0.6%, about 0.4% to about 0.5%, about 0.5% to about 1%, 0.5% to about 0.9%, 0.5% about 0.8%, about 0.5% to about 0.7%, about 0.5% to about 0.6%, about 0.6% to about 1%, 0.6% to about 0.9%, 0.6% about 0.8%, about 0.6% to about 0.7%, about 0.7% to about 1%, 0.7% to about 0.9%, 0.7% about 0.8%, about 0.8% to about 1%, 0.8% to about 0.9%, about 0.9% to about 1%, about 0.001%, about 0.01%, about 0.1%, about 0.2%, about 0.3%, about 0.4%, about 0.5%, about 0.6%, about 0.7%, about 0.8%, about 0.9%, about 1%, or any range or value therebetween by weight of the total weight of composition.

In any of the foregoing embodiments, the composition can further include a polyol. By way of example but not limitation, the composition can include a polyol that is glycerin, glycerol, or a combination thereof.

In any of the foregoing embodiments, the composition can further include a polyol, where the polyol is in an amount of about 0.01% to about 10%, about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 1.5% to about 10%, about 1.5% to about 9%, about 1.5% to about 8%, about 1.5% to about 7%, about 1.5% to about 6%, about 1.5% to about 5%, about 1.5% to about 4%, about 1.5% to about 3%, about 1.5% to about 2%, about 2% to about 10%, about 2% to about 9%, about 2% to about 8%, about 2% to about 7%, about 2% to about 6%, about 2% to about 5%, about 2% to about 4%, about 2% to about 3%, about 2.5% to about 10%, about 2.5% to about 9%, about 2.5% to about 8%, about 2.5% to about 7%, about 2.5% to about 6%, about 2.5% to about 5%, about 2.5% to about 4%, about 2.5% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, about 9% to about 10%, about 0.01%, about 0.1%, about 1%, about 1.5%, about 2%, about 2.5%, about 3%, about 4%, about 5%, about 6%, about 7%, about 8%, about 9%, about 10%, or any range or value therebetween by weight of the total weight of composition.

In any of the foregoing embodiments, the composition can further include an additive. By way of example but not limitation, the composition can include a fragrance, a colorant, an antioxidant, a buffering agent, an emollient, or a combination thereof.

In any of the foregoing embodiments, the composition can further include an additive, where the additive is in an amount of about 0.001% to about 10%, about 0.001% to about 9%, about 0.001% to about 8%, about 0.001% to about 7%, about 0.001% to about 6%, about 0.001% to about 5%, about 0.001% to about 4%, about 0.001% to about 3%, about 0.001% to about 2%, about 0.001% to about 1%, 0.01% to about 10%, about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 1.5% to about 10%, about 1.5% to about 9%, about 1.5% to about 8%, about 1.5% to about 7%, about 1.5% to about 6%, about 1.5% to about 5%, about 1.5% to about 4%, about 1.5% to about 3%, about 1.5% to about 2%, about 2% to about 10%, about 2% to about 9%, about 2% to about 8%, about 2% to about 7%, about 2% to about 6%, about 2% to about 5%, about 2% to about 4%, about 2% to about 3%, about 2.5% to about 10%, about 2.5% to about 9%, about 2.5% to about 8%, about 2.5% to about 7%, about 2.5% to about 6%, about 2.5% to about 5%, about 2.5% to about 4%, about 2.5% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, about 9% to about 10%, about 0.001%, about 0.01%, about 0.1%, about 1%, about 1.5%, about 2%, about 2.5%, about 3%, about 4%, about 5%, about 6%, about 7%, about 8%, about 9%, about 10%, or any range or value therebetween by weight of the total weight of composition.

### Personal Care Compositions

In some embodiments, a personal care composition comprises an oil of any of the foregoing embodiments of the present disclosure, including without limitation a FCO FFA oil or a MCT FFA oil, wherein the personal care composition may further comprise one or more of the following components: a fragrance, a colorant, an antioxidant, a buffering agent, an emollient, a polyol (glycerin, glycerol, or a combination thereof), a chelating agent (a tetrasubstituted sodium imino disuccinic acid, EDTA, a tetrasubstituted sodium salts of L-glutamic-N, N-diacetic acid, or any combination thereof), emulsifier (polyoxyethylene oleyl ether, PEG-40 stearate, ceteareth-12, ceteareth-30, PEG-100 stearate, methyl myristate, isopropyl myristate, glyceryl stearate, steareth-2, steareth-20, dimethicone copolyol, Polysorbate 20 (Tween 20), Polysorbate 40 (Tween 40), Polysorbate 60 (Tween 60), Polysorbate 80 (Tween 80), lauramide DEA, cocamide DEA, cocamide MEA, phospholipid PTC, alginate, carrageenans, glucate DO, methylcellulose, polyvinyl alcohol, cocamidopropyl phosphatidyl PG-dimonium chloride, stearic acid, magnesium stearate, milk amino acids, triethanolamine, magnesium aluminum silicate, or any combination thereof), a surfactant (anionic surfactant, a cationic surfactant, an amphoteric surfactant, a zwitterionic surfactant, a nonionic surfactant, or any combination thereof), an organic acid, or an organic acid salt.

The oil can be preset in the personal care composition in an amount from about 0.25% to about 10% by weight out of the total weight of the personal care composition. By way of example, but not limitation, the personal care composition can include the oil component in an amount from about 0.25% to about 4.5%, about 0.25% to about 4%, about 0.25% to about 3.5%, about 0.25% to about 3%, about 0.25% to about 2.5%, about 0.25% to about 2%, about 0.25% to about 1.5%, about 0.25% to about 1%, about 0.25% to about 0.75%, about 0.25% to about 0.5%, about 0.5% to about 5%, about 0.5% to about 4.5%, about 0.5% to about 4%, about 0.5% to about 3.5%, about 0.5% to about 3%, about 0.5% to about 2.5%, about 0.5% to about 2%, about 0.5% to about 1.5%, about 0.5% to about 1%, about 0.5% to about 0.75%, about 0.75% to about 5%, about 0.75% to about 4.5%, about 0.75% to about 4%, about 0.75% to about 3.5%, about 0.75% to about 3%, about 0.75% to about 2.5%, about 0.75% to about 2%, about 0.75% to about 1.5%, about 0.75% to about 1%, about 1% to about 5%, about 1% to about 4.5%, about 1% to about 4%, about 1% to about 3.5%, about 1% to about 3%, about 1% to about 2.5%, about 1% to about 2%, about 1% to about 1.5%, about 1.5% to about 5%, about 1.5% to about 4.5%, about 1.5% to about 4%, about 1.5% to about 3.5%, about 1.5% to about 3%, about 1.5% to about 2.5%, about 1.5% to about 2%, about 1.75% to about 5%, about 1.75% to about 4.5%, about 1.75% to about 4%, about 1.75% to about 3.5%, about 1.75% to about 3%, about 1.75% to about 2.5%, about 1.75% to about 2%, about 2% to about 5%, about 2% to about 4.5%, about 2% to about 4%, about 2% to about 3.5%, about 2% to about 3%, about 2% to about 2.5%, about 2.25% to about 5%, about 2.25% to about 4.5%, about 2.25% to about 4%, about 2.25% to about 3.5%, about 2.25% to about 3%, about 2.25% to about 2.5%, about 2.5% to about 5%, about 2.5% to about 4.5%, about 2.5% to about 4%, about 2.5% to about 3.5%, about 2.5% to about 3%, about 2.75% to about 5%, about 2.75% to about 4.5%, about 2.75% to about 4%, about 2.75% to about 3.5%, about 2.75% to about 3%, about 3% to about 5%, about 3% to about 4.5%, about 3% to about 4%, about 3% to about 3.5%, about 3.25% to about 5%, about 3.25% to about 4.5%, about 3.25% to about 4%, about 3.25% to about 3.5%, about 3.5% to about 5%, about 3.5% to about 4.5%, about 3.5% to about 4%, about 3.75% to about 5%, about 3.75% to about 4.5%, about 3.75% to about 4%, about 4% to about 5%, about 4% to about 4.5%, about 4.25% to about 5%, about 4.25% to about 4.5%, about 4.75% to about 5%, about 0.25%, 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10% or any range or value therebetween by weight out of the total weight of the personal care composition.

In any of the foregoing embodiments, the personal care composition can be in a liquid form, semi-liquid form, or a solid form. By way of example, but not limitation, the personal care composition can be in the form of a hand soap, a body wash, a face wash, a shower gel, a shampoo, a conditioner, a cleanser, an exfoliating scrub, a facial scrub, or a combination thereof.

In any of the foregoing embodiments, the personal care compositions of the present disclosure may possess an oil that provides an enhanced antimicrobial effect as compared to a personal care composition of the same formula without an oil. In one embodiment, the personal care composition comprises FFA derived from an FCO oil having a C8:C10 ratio of from about 30:70 to about 70:30 and having less than 20% C12-C18 fatty acids and the FFA is present in the personal care composition at an amount from about 2% to about 6% by weight based on the total weight of the composition and wherein the oil is at least 99% FFA by weight, based on the total weight of the oil. The personal care composition may further comprise a surfactant in an amount from about 5% to about 15% by weight of the total weight of the composition. The personal care composition of the present embodiment may provide a reduction of about 2.5 log to about 7 log in E. coli present on a contact surface and a reduction of about 2 log to about 8 log in S. aureus on a contact surface.

### Methods of Use

In some embodiments, a method is provided for using any composition or personal care composition of the present disclosure, and any of the foregoing embodiments. In any of the foregoing embodiments, the composition is added to or used as a part of a personal care composition. By way of example but not limitation, the personal care composition is applied to a subject's skin and/or hair, wet or dry, and optionally, an aqueous solution such as water is added to lather the composition and to encourage cleansing, the composition is rubbed into the skin and/or hair to further encourage cleansing, the composition is optionally left on the skin and/or hair for a desired period of time, and optionally, the composition is rinsed or removed. Optionally, the process is repeated one or two times as desired.

In some embodiments, a method of administering an antimicrobial composition to a subject in need thereof can include applying the antimicrobial composition to the subject in a therapeutically effective amount to kill microorganisms or inhibit microorganism growth. The antimicrobial compositions include any of the oil compositions described herein.

### Methods of Manufacture

Compositions of the present disclosure can be prepared by known methods. The compositions described herein are antimicrobial compositions and/or personal care compositions with antimicrobial compositions therein.

In any of the foregoing embodiments, by way of example, but not limitation, a method for making a composition includes adding an oil to the composition in an amount of about 0.25% to about 5% by weight of the total weight of the composition, wherein the oil includes free fatty acids derived from a first oil source, the first oil source having a fatty acid profile with at least 50% caprylic acid, capric acid, or a combination thereof. The method may further comprise adding free fatty acids derived from a second oil source, the second oil source comprises a fatty acid profile that is different from the fatty acid profile of the first oil source.

In any of the foregoing embodiments, by way of example but not limitation, a method for making a composition includes adding a surfactant to the composition, and adding an oil to the composition in an amount of about 0.25% to about 5% by weight of the total weight of the composition, wherein the oil includes free fatty acids derived from a first oil source, the first oil source has comprising a fatty acid profile with at least 50% caprylic acid, capric acid, or a combination thereof.

In any of the foregoing embodiments, a method for making a composition includes hydrolyzing an oil from a first oil source to provide free fatty acids and glycerol, and separating the caprylic acid and capric acid rich fraction, or a combination thereof from the glycerol and remaining free fatty acids.

In any of the foregoing embodiments, hydrolyzing oil from the first oil source to provide free fatty acids and glycerol includes a hydrolysis step. By way of example but not limitation, FIG. 1 illustrates a method of making a fractionated coconut oil, which includes a process for hydrolyzing an oil from a first oil source to provide free fatty acids and glycerol. In such non-limiting example, crude coconut oil is hydrolyzed by an enzyme process to separating FFA from glycerol. In FIG. 1 or 1C, the starting oil is coconut oil.

Hydrolyzing the oil from a first oil source to provide free fatty acids and glycerol includes hydrolyzing triacylglycerides (TAGs) in the starting oil. By way of example but not limitation, the hydrolysis of TAGs in the starting oil may be carried out by a lipase such as lipase AY (Amano Enzymes, USA Elgin IL, USA), or any non-regiospecific lipase that cuts at the sn-1, sn-2 and sn-3 positions.

In some embodiments, the hydrolyzing TAGs in the starting oil may be carried out at a temperature of about 30° C. to 60° C. By way of example but not limitation, the first step of hydrolyzing TAGs in the starting oil may be carried out at a temperature of 30° C. to 35° C., 31° C. to 35° C., 32° C. to 35° C., 33° C. to 35° C., 34° C. to 35° C., 30° C. to 34° C., 31° C. to 34° C., 32° C. to 34° C., 33° C. to 34° C., 30° C. to 33° C., 31° C. to 33° C., 32° C. to 33° C., 30° C. to 32° C., 31° C. to 32° C., 30° C. to 31° C., or 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., or any range or value therebetween.

In some embodiments, the first step of hydrolyzing TAGs in the starting oil may be carried out for about 14 hours to 24 hours. By way of example but not limitation, the first step of hydrolyzing TAGs in the starting oil may be carried out for 14 hours to 20 hours, 14 hours to 16 hours, 18 hours to 24 hours, 22 hours to 24 hours, 18 hours to 20 hours, or about 14 hours, 15 hours, 16 hours, 17 hours, 18 hours, 19 hours, 20 hours, 21 hours, 22 hours, 23 hours, or 24 hours, or any range or value therebetween.

In some embodiments, the first step of hydrolyzing TAGs in the starting oil results in hydrolysis of substantially all TAG. By way of example but not limitation, the hydrolyzing TAGs in the starting oil results in hydrolysis of 94% to 100%, 95% to 100%, 96% to 100%, 97% to 100%, 98% to 100%, 99% to 100%, 94% to 99%, 95% to 99%, 96% to 99%, 97% to 99%, 98% to 99%, 94% to 98%, 95% to 98%, 96% to 98%, 97% to 98%, 94% to 97%, 95% to 97%, 96% to 97%, 94% to 96%, 95% to 96%, or 94% to 95% TAG, or at least 94%, 95%, 96%, 97%, 98%, 99%, or 100% of TAG.

After hydrolyzing the starting oil, crude glycerol is separated from a mixture of free fatty acids.

In one or more embodiment, as shown in FIG. 1 (3A), winterization technology can be employed to fractionate a capric and caprylic fatty acid-rich fraction.

In any one of the foregoing embodiments, distillation or fractionation, as shown in FIG. 1 (3B), is performed after hydrolyzing the starting oil to separate capric acid, caprylic acid, or a combination thereof (medium chain fatty acids) from remaining long chain free fatty acids. By removing the higher molecular long chain free fatty acids, the resulting oil is liquid at room temperatures.

In some embodiments, the process as shown in FIG. 1 (4) further includes reacting the caprylic acid, the capric acid, or the combination thereof with glycerol to form a composition comprising a caprylic and capric-rich triglyceride fraction. (TAGs). Such reaction includes a re-esterification process to enrich the TAG oil content, in particular with caprylic-capric triglycerides. Processes such as these are used to produce Medium Chain Triglycerides (MCT) oil and Fractionated Coconut Oil.

In any of the foregoing embodiments, the process of making a product enriched in caprylic-capric triglycerides results in forming a fractionated coconut oil (FCO).

In any of the foregoing embodiments, the enriched in caprylic-capric triglycerides, which can be FCO, is further hydrolyzed to form enriched caprylic acid, capric acid, or a combination there of free fatty acids. By way of example but not limitation, the hydrolysis of caprylic-capric triglycerides may be carried out by physical or chemical means or by a lipase such as lipase AY (Amano Enzymes, USA Elgin IL, USA), or any non-regiospecific lipase that cuts at the sn-1, sn-2 and sn-3 positions.

In some embodiments, the hydrolyzing caprylic-capric triglycerides may be carried out at a temperature of about 30° C. to 65° C. By way of example but not limitation, the first step of hydrolyzing TAGs in the starting oil may be carried out at a temperature of 30° C. to 35° C., 31° C. to 35° C., 32° C. to 35° C., 33° C. to 35° C., 34° C. to 35° C., 30° C. to 34° C., 31° C. to 34° C., 32° C. to 34° C., 33° C. to 34° C., 30° C. to 33° C., 31° C. to 33° C., 32° C. to 33° C., 30° C. to 32° C., 31° C. to 32° C., 30° C. to 31° C., or 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., and 65° C.

In some embodiments, the first step of hydrolyzing TAGs in the starting oil may be carried out for about 14 hours to 24 hours. By way of example but not limitation, the first step of hydrolyzing caprylic-capric triglycerides in the starting oil may be carried out for 14 hours to 20 hours, 14 hours to 16 hours, 18 hours to 24 hours, 22 hours to 24 hours, 18 hours to 20 hours, or about 14 hours, 15 hours, 16 hours, 17 hours, 18 hours, 19 hours, 20 hours, 21 hours, 22 hours, 23 hours, or 24 hours.

In some embodiments, the first step of hydrolyzing caprylic-capric triglycerides results in hydrolysis of substantially all TAG. By way of example but not limitation, the hydrolyzing TAGs in the starting oil results in hydrolysis of 94% to 100%, 95% to 100%, 96% to 100%, 97% to 100%, 98% to 100%, 99% to 100%, 94% to 99%, 95% to 99%, 96% to 99%, 97% to 99%, 98% to 99%, 94% to 98%, 95% to 98%, 96% to 98%, 97% to 98%, 94% to 97%, 95% to 97%, 96% to 97%, 94% to 96%, 95% to 96%, or 94% to 95% TAG, or at least 94%, 95%, 96%, 97%, 98%, 99%, or 100% of TAG.

In any of the foregoing embodiments, the methods of making compositions include adding free fatty acids derived from a second oil source to the composition, where the second oil source comprises a second fatty acid profile that is different from the first fatty acid profile. In any of the foregoing embodiments, the methods further include adding one or more of a surfactant, polyol, chelating agent, organic acid or salt thereof, or additive.

Additional embodiments of the present disclosure include the following list, which is not intended to be an exhaustive list of all possible embodiments, but rather a list of preferred embodiments.

First Embodiment: A composition comprising an oil in an amount of about 0.25% to about 10% by weight of the total weight of the composition, the oil comprising free fatty acids derived from a first oil source, the first oil source comprising a fatty acid profile comprising at least 50% caprylic acid, capric acid, or a combination thereof.

The composition of any one of or any combination of any of the applicable preceding first embodiments, wherein the first oil source is a fractionated coconut oil.

The composition of any one of or any combination of any of the applicable preceding first embodiments, wherein the first oil source is a fractionated coconut oil having a C8:C10 ratio of from about 30:70 to about 70:30 and more preferably from about 40:60 to about 60:40.

The composition of any one of or any combination of any of the applicable preceding first embodiments, wherein the oil is present in an about from about 2% to about 7% by weight based on the total weight of the composition.

The composition of any one of or any combination of any of the applicable preceding first embodiments, wherein the first source oil has less than 20% C12-C16 fatty acids and more preferably less than 1% C12-C16 fatty acids.

The composition of any one of or any combination of any of the applicable preceding first embodiments, wherein the oil comprises at least 99% FFA.

The composition of any one of or any combination of any of the applicable preceding first embodiments further comprising a surfactant present in an amount from about 5% to about 15% by weight of the total weight of the composition.

The composition of any one of or any combination of any of the applicable preceding first embodiments, wherein the composition comprises about a 2 log to about a 7 fold log reduction in a bacteria, including E. coli. Or S. aureus, on a contact surface as compared to the composition without the oil.

The composition of any one of or any combination of any of the applicable preceding first embodiments, wherein the composition is a body wash or a hand soap.

The composition of any one of or any combination of any of the applicable preceding first embodiments further comprising free fatty acids derived from a second oil source, the second oil source comprises a fatty acid profile that is different from the fatty acid profile of the first oil source.

The composition of any one of or any combination of any of the applicable preceding first embodiments, wherein the free fatty acids derived from the first oil source are from about 10% to about 99% by weight of the total weight of the oil.

The composition of any one of or any combination of any of the applicable preceding first embodiments, wherein the first oil source is coconut oil.

The composition of any one of or any combination of any of the applicable preceding first embodiments, wherein the free fatty acids derived from the second oil source are from about 1% to about 90% of the total weight of the oil.

The composition of any one of or any combination of any of the applicable preceding first embodiments, wherein the second oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or combinations thereof.

The composition of any one of or any combination of any of the applicable preceding first embodiments, wherein the second oil source is flaxseed oil.

The composition of any one of or any combination of any of the applicable preceding first embodiments, wherein the oil further comprises a mixture of monoacylglycerides, diacylglycerides, and free fatty acids derived from a third oil source, the third oil source comprises a fatty acid profile that is different from each of the fatty acid profiles of the first oil source and second oil source.

The composition of any one of or any combination of any of the applicable preceding first embodiments, wherein the mixture of monoacylglycerides, diacylglycerides, and free fatty acids derived from the third oil source are about 1% to about 80% by weight of the total weight of the oil.

The composition of any one of or any combination of any of the applicable preceding first embodiments, wherein the third oil source is an enzyme-modified oil.

The composition of any one of or any combination of any of the applicable preceding first embodiments, wherein the third oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or any combination thereof.

The composition of any one of or any combination of any of the applicable preceding first embodiments, wherein the third oil source is oat oil.

The composition of any one of or any combination of any of the applicable preceding first embodiments, wherein the monoacylglycerides are about 60% to about 95% by weight of the total weight of the mixture of monoacylglycerides, diacylglycerides, and free fatty acids derived from the third oil source.

The composition of any one of or any combination of any of the applicable preceding first embodiments, wherein the free fatty acids are about 5% to about 30% by weight of the total weight of the mixture of monoacylglycerides, diacylglycerides, and free fatty acids derived from the third oil source.

The composition of any one of or any combination of any of the applicable preceding first embodiments, wherein the mixture of monoacylglycerides and free fatty acids derived from the third oil source further comprises diacylglycerides derived from the third oil source.

The composition of any one of or any combination of any of the applicable preceding first embodiments, wherein the diacylglycerides are about 1% to about 20% by weight of the total weight of the mixture of monoacylglycerides, free fatty acids, and diacylglycerides derived from the third oil source.

The composition of any one of or any combination of any of the applicable preceding first embodiments, further comprising an organic acid, an organic acid salt, or a combination thereof.

The composition of any one of or any combination of any of the applicable preceding first embodiments, wherein the organic acid is lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The composition of any one of or any combination of any of the applicable preceding first embodiments, wherein the organic acid salt is a salt of lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The composition of any one of or any combination of any of the applicable preceding first embodiments, where the organic acid salt is a sodium salt or a potassium salt of benzoic acid.

The composition of any one of or any combination of any of the applicable preceding first embodiments, wherein the organic acid, organic acid salt, or combination thereof is about 0.001 to about 10% of the total weight of the composition.

The composition of any one of or any combination of any of the applicable preceding first embodiments, wherein the composition is a personal care composition.

The composition of any one of or any combination of any of the applicable preceding first embodiments, wherein the personal care composition is a hand soap, a body wash, a face wash, a shower gel, a shampoo, a conditioner, a cleanser, an exfoliating scrub, a facial scrub, or a combination thereof.

The composition of any one of or any combination of any of the applicable preceding first embodiments, further comprising a surfactant in an amount of about 1% to about 50% of the total weight of the composition.

The composition of any one of or any combination of any of the applicable preceding first embodiments, wherein the surfactant is an anionic surfactant, a cationic surfactant, an amphoteric surfactant, a nonionic surfactant, or any combination thereof.

The composition of any one of or any combination of any of the applicable preceding first embodiments, further comprising a chelating agent.

The composition of any one of or any combination of any of the applicable preceding first embodiments, wherein the chelating agent is about 0.001% to about 1% of the total weigh of the composition.

The composition of any one of or any combination of any of the applicable preceding first embodiments, further comprising a polyol.

The composition of any one of or any combination of any of the applicable preceding first embodiments, wherein the polyol is about 0.01 to about 10% of the total weight of the composition.

The composition of any one of or any combination of any of the applicable preceding first embodiments, further comprising one or more additives.

The composition of any one of or any combination of any of the applicable preceding first embodiments, wherein the one or more additives is a fragrance, a colorant, an antioxidant, a buffering agent, an emollient, or a combination thereof.

The composition of any one of or any combination of any of the applicable preceding first embodiments, wherein each of the additives is about 0.001% to about 10% of the total weight of the composition.

Second Embodiment: A composition comprising a surfactant and an oil in an amount of about 0.25% to about 5% by weight of the total weight of the composition, the oil comprising free fatty acids derived from a first oil source, the first oil source comprising a fatty acid profile comprising at least 50% caprylic acid, capric acid, or a combination thereof.

The composition of any one of or any combination of any of the applicable preceding second embodiments, wherein the oil further comprises free fatty acids derived from a second oil source, the second oil source comprises a fatty acid profile that is different from the fatty acid profile of the first oil source.

The composition of any one of or any combination of any of the applicable preceding second embodiments, wherein the free fatty acids derived from the first oil source are from about 10% to about 99% by weight of the total weight of the oil.

The composition of any one of or any combination of any of the applicable preceding second embodiments, wherein the first oil source is coconut oil.

The composition of any one of or any combination of any of the applicable preceding second embodiments, wherein the free fatty acids derived from the second oil source are from about 1% to about 90% of the total weight of the oil.

The composition of any one of or any combination of any of the applicable preceding second embodiments, wherein the second oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or combinations thereof.

The composition of any one of or any combination of any of the applicable preceding second embodiments, wherein the second oil source is flaxseed oil.

The composition of any one of or any combination of any of the applicable preceding second embodiments, wherein the oil further comprises a mixture of monoacylglycerides, diacylglycerides, and free fatty acids derived from a third oil source, the third oil source comprises a fatty acid profile that is different from each of the fatty acid profiles of the first oil source and second oil source.

The composition of any one of or any combination of any of the applicable preceding second embodiments, wherein the mixture of monoacylglycerides, diacylglycerides, and free fatty acids derived from the third oil source are about 1% to about 80% by weight of the total weight of the oil.

The composition of any one of or any combination of any of the applicable preceding second embodiments, wherein the third oil source is an enzyme-modified oil.

The composition of any one of or any combination of any of the applicable preceding second embodiments, wherein the third oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or any combination thereof.

The composition of any one of or any combination of any of the applicable preceding second embodiments, wherein the third oil source is oat oil.

The composition of any one of or any combination of any of the applicable preceding second embodiments, wherein the monoacylglycerides are about 60% to about 95% by weight of the total weight of the mixture of monoacylglycerides, diacylglycerides, and free fatty acids derived from the third oil source.

The composition of any one of or any combination of any of the applicable preceding second embodiments, wherein the free fatty acids are about 5% to about 30% by weight of the total weight of the mixture of monoacylglycerides, diacylglycerides, and free fatty acids derived from the third oil source.

The composition of any one of or any combination of any of the applicable preceding second embodiments, wherein the mixture of monoacylglycerides and free fatty acids derived from the third oil source further comprises diacylglycerides derived from the third oil source.

The composition of any one of or any combination of any of the applicable preceding second embodiments, wherein the diacylglycerides are about 1% to about 20% by weight of the total weight of the mixture of monoacylglycerides, free fatty acids, and diacylglycerides derived from the third oil source.

The composition of any one of or any combination of any of the applicable preceding second embodiments, further comprising an organic acid, an organic acid salt, or a combination thereof.

The composition of any one of or any combination of any of the applicable preceding second embodiments, wherein the organic acid is lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The composition of any one of or any combination of any of the applicable preceding second embodiments, wherein the organic acid salt is a salt of lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The composition of any one of or any combination of any of the applicable preceding second embodiments, where the organic acid salt is a sodium salt or a potassium salt of benzoic acid.

The composition of any one of or any combination of any of the applicable preceding second embodiments, wherein the organic acid, organic acid salt, or combination thereof is about 0.001 to about 10% of the total weight of the composition.

The composition of any one of or any combination of any of the applicable preceding second embodiments, wherein the composition is a personal care composition.

The composition of any one of or any combination of any of the applicable preceding second embodiments, wherein the personal care composition is a hand soap, a body wash, a face wash, a shower gel, a shampoo, a conditioner, a cleanser, an exfoliating scrub, a facial scrub, or a combination thereof.

The composition of any one of or any combination of any of the applicable preceding second embodiments, further comprising a surfactant in an amount of about 1% to about 50% of the total weight of the composition.

The composition of any one of or any combination of any of the applicable preceding second embodiments, wherein the surfactant is an anionic surfactant, a cationic surfactant, an amphoteric surfactant, a nonionic surfactant, or any combination thereof.

The composition of any one of or any combination of any of the applicable preceding second embodiments, further comprising a chelating agent.

The composition of any one of or any combination of any of the applicable preceding second embodiments, wherein the chelating agent is about 0.001% to about 1% of the total weigh of the composition.

The composition of any one of or any combination of any of the applicable preceding second embodiments, further comprising a polyol.

The composition of any one of or any combination of any of the applicable preceding second embodiments, wherein the polyol is about 0.01 to about 10% of the total weight of the composition.

The composition of any one of or any combination of any of the applicable preceding second embodiments further comprising one or more additives.

The composition of any one of or any combination of any of the applicable preceding second embodiments, wherein the one or more additives is a fragrance, a colorant, an antioxidant, a buffering agent, an emollient, or a combination thereof.

The composition of any one of or any combination of any of the applicable preceding second embodiments, wherein each of the additives is about 0.001% to about 10% of the total weight of the composition.

Third Embodiment: A method for making a composition, the method comprising adding an oil to the composition in an amount of about 0.25% to about 5% by weight of the total weight of the composition, the oil comprising free fatty acids derived from a first oil source, the first oil source comprising a fatty acid profile comprising at least 50% caprylic acid, capric acid, or a combination thereof.

The method of any one of or any combination of any of the applicable preceding third embodiments further comprising free fatty acids derived from a second oil source, the second oil source comprises a fatty acid profile that is different from the fatty acid profile of the first oil source.

The method of any one of or any combination of any of the applicable preceding third embodiments, wherein the free fatty acids derived from the first oil source are from about 10% to about 99% by weight of the total weight of the oil.

The method of any one of or any combination of any of the applicable preceding third embodiments, wherein the first oil source is coconut oil.

The method of any one of or any combination of any of the applicable preceding third embodiments, wherein the free fatty acids derived from the second oil source are from about 1% to about 90% of the total weight of the oil.

The method of any one of or any combination of any of the applicable preceding third embodiments, wherein the second oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or combinations thereof.

The method of any one of or any combination of any of the applicable preceding third embodiments, wherein the second oil source is flaxseed oil.

The method of any one of or any combination of any of the applicable preceding third embodiments, wherein the oil further comprises a mixture of monoacylglycerides, diacylglycerides, and free fatty acids derived from a third oil source, the third oil source comprises a fatty acid profile that is different from each of the fatty acid profiles of the first oil source and second oil source.

The method of any one of or any combination of any of the applicable preceding third embodiments, wherein the mixture of monoacylglycerides, diacylglycerides, and free fatty acids derived from the third oil source are about 1% to about 80% by weight of the total weight of the oil.

The method of any one of or any combination of any of the applicable preceding third embodiments, wherein the third oil source is an enzyme-modified oil.

The method of any one of or any combination of any of the applicable preceding third embodiments, wherein the third oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or any combination thereof.

The method of any one of or any combination of any of the applicable preceding third embodiments, wherein the third oil source is oat oil.

The method of any one of or any combination of any of the applicable preceding third embodiments, wherein the monoacylglycerides are about 60% to about 95% by weight of the total weight of the mixture of monoacylglycerides, diacylglycerides, and free fatty acids derived from the third oil source.

The method of any one of or any combination of any of the applicable preceding third embodiments, wherein the free fatty acids are about 5% to about 30% by weight of the total weight of the mixture of monoacylglycerides, diacylglycerides, and free fatty acids derived from the third oil source.

The method of any one of or any combination of any of the applicable preceding third embodiments, wherein the mixture of monoacylglycerides and free fatty acids derived from the third oil source further comprises diacylglycerides derived from the third oil source.

The method of any one of or any combination of any of the applicable preceding third embodiments, wherein the diacylglycerides are about 1% to about 20% by weight of the total weight of the mixture of monoacylglycerides, free fatty acids, and diacylglycerides derived from the third oil source.

The method of any one of or any combination of any of the applicable preceding third embodiments, wherein the composition further comprises an organic acid, an organic acid salt, or a combination thereof.

The method of any one of or any combination of any of the applicable preceding third embodiments, wherein the organic acid is lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The method of any one of or any combination of any of the applicable preceding third embodiments, wherein the organic acid salt is a salt of lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The method of any one of or any combination of any of the applicable preceding third embodiments, where the organic acid salt is a sodium salt or a potassium salt of benzoic acid.

The method of any one of or any combination of any of the applicable preceding third embodiments, wherein the organic acid, organic acid salt, or combination thereof is about 0.001 to about 10% of the total weight of the composition.

The method of any one of or any combination of any of the applicable preceding third embodiments, wherein the composition is a personal care composition.

The method of any one of or any combination of any of the applicable preceding third embodiments, wherein the personal care composition is a hand soap, a body wash, a face wash, a shower gel, a shampoo, a conditioner, a cleanser, an exfoliating scrub, a facial scrub, or a combination thereof.

The method of any one of or any combination of any of the applicable preceding third embodiments, wherein the composition further comprises a surfactant in an amount of about 1% to about 50% of the total weight of the composition.

The method of any one of or any combination of any of the applicable preceding third embodiments, wherein the surfactant is an anionic surfactant, a cationic surfactant, an amphoteric surfactant, a nonionic surfactant, or any combination thereof.

The method of any one of or any combination of any of the applicable preceding third embodiments, wherein the composition further comprises a chelating agent.

The method of any one of or any combination of any of the applicable preceding third embodiments, wherein the chelating agent is about 0.001% to about 1% of the total weigh of the composition.

The method of any one of or any combination of any of the applicable preceding third embodiments, wherein the composition further comprises a polyol.

The method of any one of or any combination of any of the applicable preceding third embodiments, wherein the polyol is about 0.01 to about 10% of the total weight of the composition.

The method of any one of or any combination of any of the applicable preceding third embodiments, wherein the composition further comprises one or more additives.

The method of any one of or any combination of any of the applicable preceding third embodiments, wherein the one or more additives is a fragrance, a colorant, an antioxidant, a buffering agent, an emollient, or a combination thereof.

The method of any one of or any combination of any of the applicable preceding third embodiments, wherein each of the additives is about 0.001% to about 10% of the total weight of the composition.

Fourth Embodiment: A method for making a composition, the method comprising adding a surfactant to the composition and adding an oil to the composition in an amount of about 0.25% to about 5% by weight of the total weight of the composition, the oil comprising free fatty acids derived from a first oil source, the first oil source comprising a fatty acid profile comprising at least 50% caprylic acid, capric acid, or a combination thereof.

The method of any one of or any combination of any of the applicable preceding fourth embodiments, wherein the oil further comprises free fatty acids derived from a second oil source, the second oil source comprises a fatty acid profile that is different from the fatty acid profile of the first oil source.

The method of any one of or any combination of any of the applicable preceding fourth embodiments, wherein the free fatty acids derived from the first oil source are from about 10% to about 99% by weight of the total weight of the oil.

The method of any one of or any combination of any of the applicable preceding fourth embodiments, wherein the first oil source is coconut oil.

The method of any one of or any combination of any of the applicable preceding fourth embodiments, wherein the free fatty acids derived from the second oil source are from about 1% to about 90% of the total weight of the oil.

The method of any one of or any combination of any of the applicable preceding fourth embodiments, wherein the second oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or combinations thereof.

The method of any one of or any combination of any of the applicable preceding fourth embodiments, wherein the second oil source is flaxseed oil.

The method of any one of or any combination of any of the applicable preceding fourth embodiments, wherein the oil further comprises a mixture of monoacylglycerides, diacylglycerides, and free fatty acids derived from a third oil source, the third oil source comprises a fatty acid profile that is different from each of the fatty acid profiles of the first oil source and second oil source.

The method of any one of or any combination of any of the applicable preceding fourth embodiments, wherein the mixture of monoacylglycerides, diacylglycerides, and free fatty acids derived from the third oil source are about 1% to about 80% by weight of the total weight of the oil.

The method of any one of or any combination of any of the applicable preceding fourth embodiments, wherein the third oil source is an enzyme-modified oil.

The method of any one of or any combination of any of the applicable preceding fourth embodiments, wherein the third oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or any combination thereof.

The method of any one of or any combination of any of the applicable preceding fourth embodiments, wherein the third oil source is oat oil.

The method of any one of or any combination of any of the applicable preceding fourth embodiments, wherein the monoacylglycerides are about 60% to about 95% by weight of the total weight of the mixture of monoacylglycerides, diacylglycerides, and free fatty acids derived from the third oil source.

The method of any one of or any combination of any of the applicable preceding fourth embodiments, wherein the free fatty acids are about 5% to about 30% by weight of the total weight of the mixture of monoacylglycerides, diacylglycerides, and free fatty acids derived from the third oil source.

The method of any one of or any combination of any of the applicable preceding fourth embodiments, wherein the mixture of monoacylglycerides and free fatty acids derived from the third oil source further comprises diacylglycerides derived from the third oil source.

The method of any one of or any combination of any of the applicable preceding fourth embodiments, wherein the diacylglycerides are about 1% to about 20% by weight of the total weight of the mixture of monoacylglycerides, free fatty acids, and diacylglycerides derived from the third oil source.

The method of any one of or any combination of any of the applicable preceding fourth embodiments, wherein the composition further comprises an organic acid, an organic acid salt, or a combination thereof.

The method of any one of or any combination of any of the applicable preceding fourth embodiments, wherein the organic acid is lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The method of any one of or any combination of any of the applicable preceding fourth embodiments, wherein the organic acid salt is a salt of lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The method of any one of or any combination of any of the applicable preceding fourth embodiments, where the organic acid salt is a sodium salt or a potassium salt of benzoic acid.

The method of any one of or any combination of any of the applicable preceding fourth embodiments, wherein the organic acid, organic acid salt, or combination thereof is about 0.001 to about 10% of the total weight of the composition.

The method of any one of or any combination of any of the applicable preceding fourth embodiments, wherein the composition is a personal care composition.

The method of any one of or any combination of any of the applicable preceding fourth embodiments, wherein the personal care composition is a hand soap, a body wash, a face wash, a shower gel, a shampoo, a conditioner, a cleanser, an exfoliating scrub, a facial scrub, or a combination thereof.

The method of any one of or any combination of any of the applicable preceding fourth embodiments, wherein the composition further comprises a surfactant in an amount of about 1% to about 50% of the total weight of the composition.

The method of any one of or any combination of any of the applicable preceding fourth embodiments, wherein the surfactant is an anionic surfactant, a cationic surfactant, an amphoteric surfactant, a nonionic surfactant, or any combination thereof.

The method of any one of or any combination of any of the applicable preceding fourth embodiments, wherein the composition further comprises a chelating agent.

The method of any one of or any combination of any of the applicable preceding fourth embodiments, wherein the chelating agent is about 0.001% to about 1% of the total weigh of the composition.

The method of any one of or any combination of any of the applicable preceding fourth embodiments, wherein the composition further comprises a polyol.

The method of any one of or any combination of any of the applicable preceding fourth embodiments, wherein the polyol is about 0.01 to about 10% of the total weight of the composition.

The method of any one of or any combination of any of the applicable preceding fourth embodiments, wherein the composition further comprises one or more additives.

The method of any one of or any combination of any of the applicable preceding fourth embodiments, wherein the one or more additives is a fragrance, a colorant, an antioxidant, a buffering agent, an emollient, or a combination thereof.

The method of any one of or any combination of any of the applicable preceding fourth embodiments, wherein each of the additives is about 0.001% to about 10% of the total weight of the composition.

Fifth Embodiment: A method for making a composition, the method comprising hydrolyzing an oil from a first oil source to provide free fatty acids and glycerol and separating caprylic acid, the capric acid, or a combination thereof from the glycerol and remaining free fatty acids, wherein the separating caprylic acid, capric acid, or a combination thereof comprises a winterization process.

The method of any one of or any combination of any of the applicable preceding fifth embodiments, further comprising isolating a fraction comprising the caprylic acid, the capric acid, or the combination thereof.

The method of any one of or any combination of any of the applicable preceding fifth embodiments, wherein the hydrolyzing comprises an enzyme-mediated hydrolysis.

The method of any one of or any combination of any of the applicable preceding fifth embodiments, further comprising adding free fatty acids derived from a second oil source to the composition, the second oil source comprises a second fatty acid profile that is different from the first fatty acid profile.

The method of any one of or any combination of any of the applicable preceding fifth embodiments, further comprising adding a surfactant to the composition.

T The method of any one of or any combination of any of the applicable preceding fifth embodiments, wherein the free fatty acids derived from the first oil source are from about 10% to about 99% by weight of the total weight of the oil.

The method of any one of or any combination of any of the applicable preceding fifth embodiments, wherein the first oil source is coconut oil.

The method of any one of or any combination of any of the applicable preceding fifth embodiments, wherein the free fatty acids derived from the second oil source are from about 1% to about 90% of the total weight of the oil.

The method of any one of or any combination of any of the applicable preceding fifth embodiments, wherein the second oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or combinations thereof.

The method of any one of or any combination of any of the applicable preceding fifth embodiments, wherein the second oil source is flaxseed oil.

The method of any one of or any combination of any of the applicable preceding fifth embodiments, wherein the oil further comprises a mixture of monoacylglycerides, diacylglycerides, and free fatty acids derived from a third oil source, the third oil source comprises a fatty acid profile that is different from each of the fatty acid profiles of the first oil source and second oil source.

The method of any one of or any combination of any of the applicable preceding fifth embodiments, wherein the mixture of monoacylglycerides, diacylglycerides, and free fatty acids derived from the third oil source are about 1% to about 80% by weight of the total weight of the oil.

The method of any one of or any combination of any of the applicable preceding fifth embodiments, wherein the third oil source is an enzyme-modified oil.

The method of any one of or any combination of any of the applicable preceding fifth embodiments, wherein the third oil source is olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or any combination thereof.

The method of any one of or any combination of any of the applicable preceding fifth embodiments, wherein the third oil source is oat oil.

The method of any one of or any combination of any of the applicable preceding fifth embodiments, wherein the monoacylglycerides are about 60% to about 95% by weight of the total weight of the mixture of monoacylglycerides, diacylglycerides, and free fatty acids derived from the third oil source.

The method of any one of or any combination of any of the applicable preceding fifth embodiments, wherein the free fatty acids are about 5% to about 30% by weight of the total weight of the mixture of monoacylglycerides, diacylglycerides, and free fatty acids derived from the third oil source.

The method of any one of or any combination of any of the applicable preceding fifth embodiments, wherein the mixture of monoacylglycerides and free fatty acids derived from the third oil source further comprises diacylglycerides derived from the third oil source.

The method of any one of or any combination of any of the applicable preceding fifth embodiments, wherein the diacylglycerides are about 1% to about 20% by weight of the total weight of the mixture of monoacylglycerides, free fatty acids, and diacylglycerides derived from the third oil source.

The method of any one of or any combination of any of the applicable preceding fifth embodiments, wherein the composition further comprises an organic acid, an organic acid salt, or a combination thereof.

The method of any one of or any combination of any of the applicable preceding fifth embodiments, wherein the organic acid is lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The method of any one of or any combination of any of the applicable preceding fifth embodiments, wherein the organic acid salt is a salt of lactic acid, tartaric acid, adipic acid, succinic acid, ascorbic acid, malonic acid, oxalic acid, pyruvic acid, picolinic acid, dipicolinic acid, citric acid, benzoic acid, formic acid, acetic acid, propionic acid, or any combination thereof.

The method of any one of or any combination of any of the applicable preceding fifth embodiments, where the organic acid salt is a sodium salt or a potassium salt of benzoic acid.

The method of any one of or any combination of any of the applicable preceding fifth embodiments, wherein the organic acid, organic acid salt, or combination thereof is about 0.001 to about 10% of the total weight of the composition.

The method of any one of or any combination of any of the applicable preceding fifth embodiments, wherein the composition is a personal care composition.

The method of any one of or any combination of any of the applicable preceding fifth embodiments, wherein the personal care composition is a hand soap, a body wash, a face wash, a shower gel, a shampoo, a conditioner, a cleanser, an exfoliating scrub, a facial scrub, or a combination thereof.

The method of any one of or any combination of any of the applicable preceding fifth embodiments, wherein the composition further comprises a surfactant in an amount of about 1% to about 50% of the total weight of the composition.

The method of any one of or any combination of any of the applicable preceding fifth embodiments, wherein the surfactant is an anionic surfactant, a cationic surfactant, an amphoteric surfactant, a nonionic surfactant, or any combination thereof.

The method of any one of or any combination of any of the applicable preceding fifth embodiments, wherein the composition further comprises a chelating agent.

The method of any one of or any combination of any of the applicable preceding fifth embodiments, wherein the chelating agent is about 0.001% to about 1% of the total weigh of the composition.

The method of any one of or any combination of any of the applicable preceding fifth embodiments, wherein the composition further comprises a polyol.

The method of any one of or any combination of any of the applicable preceding fifth embodiments, wherein the polyol is about 0.01 to about 10% of the total weight of the composition.

The method of any one of or any combination of any of the applicable preceding fifth embodiments, wherein the composition further comprises one or more additives.

The method of any one of or any combination of any of the applicable preceding fifth embodiments, wherein the one or more additives is a fragrance, a colorant, an antioxidant, a buffering agent, an emollient, or a combination thereof.

The method of any one of or any combination of any of the applicable preceding fifth embodiments, wherein each of the additives is about 0.001% to about 10% of the total weight of the composition.

### EXAMPLES

In the following examples, unless otherwise noted, the MAG, DAG, TAG and FFA content and fatty acid profiles can be as described in the above tables or in the foregoing tables.

Different source oils are abbreviated as follows: Algae: Ag; Almond: Al; Coconut: Co; Fish: F; Flaxseed: Fx; Hempseed: H; MCT: M; Oat: Oa; Olive: O; Rosehip: K Sesame.

The shorthand for each composition is derived as follows: Source of EMO/Source of FFA-rich oil 1 and percentage in the mixture/source of the FFA-rich oil 2 and percentage in the mixture. For example, a 50% Flaxseed EMO oil and 50% Flaxseed FFA would be simply Fx/Fx-50; a mixture of 25% Coconut EMO and 75% Rosehip FFA would be Co/R-75.

Formulations produced as oil-in-water nanoemulsions are prefixed with an “n.” For example nCo/R-27 is a nanoemulsion produced from an oil that is 25% coconut EMO and 75% Rosehip FFA.

Preparation of 1:9 Oil/Water nanoemulsions: Stocks of EMO and solution were kept in a 4° C. refrigerator (at which temperature the oils solidify), in amber bottles topped with nitrogen to prevent exposure to light and oxygen. Prior to use, aliquots of oil solutions were placed in a 60° C. water bath to melt, and kept at 60° C. To a 100 mL stainless-steel cup, 13.5 g of water was added and warmed in the 60° C. water bath. 1.5 g of total oil was added to the water (for example, 0.75 g MAG, 0.75 FFA for 50/50 MAG/FFA ratio) and immediately homogenized using a Brinkmann 110V homogenizer for 1 minute with manual rotation to ensure evenness. The sample was then sonicated using a QSonica at 90% amplitude with 5 minutes On and, 1 minute Off, three times, for a total of 15 minutes On. Aliquots of each 5 min were diluted 1:1000 in water and analyzed on a Malvern Zetasizer for Z-avg, PDI, and zeta potential. Analysis was done with three consecutive reading replicates conducted. Nanoemulsions were aliquoted and stored both at room temperature in the dark, and in a 4° C. refrigerator in the dark.

### Example 1: Free Fatty Acid (FFA) Derived from First Oil Source

Free fatty acids (FFA) were prepared by the complete hydrolysis of TAGs in a starting coconut oil to FFA and glycerol followed by subsequent separation of the glycerol from the FFA oil. FFA oils were prepared in a 5 L stirred, jacketed glass reactor. Citric acid buffer (100 mM, pH 5.8) was prepared by combining 24 grams of food grade citric acid with 13 g of food grade sodium hydroxide in 1235 mL of distilled de-ionized water. Amano Lipase AY (0.5 g) was dissolved in the 25 mL solution, which was then heated to 33° C. and agitated at 400 rpm. After a brief (10 min.) vacuum degassing, 1,111 mL of starting oil was added to the reactor. The reaction volume was overlaid with nitrogen and the reaction proceeded for 12-18 hours until all of the TAGs had been converted to fatty acids and glycerol. Once the reaction was complete, the mixture was heated to 70° C. for 1 hour to inactivate the lipase. Agitation was stopped. After the two phases had separated, the upper phase (the free fatty acid oil) was removed and stored at 4° C. in opaque plastic bottles under nitrogen.

Components of the oil samples were analyzed using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed.

### Example 2: Free Fatty Acid (FFA) Derived from Second Oil Source

Free fatty acids derived from a second oil source were prepared by the complete hydrolysis of TAGs in a starting flaxseed oil to FFA and glycerol followed by subsequent separation of the glycerol from the FFA oil. FFA oils were prepared in a 5 L stirred, jacketed glass reactor. Citric acid buffer (100 mM, pH 5.8) was prepared by combining 24 grams of food grade citric acid with 13 g of food grade sodium hydroxide in 1235 mL of distilled de-ionized water. Amano Lipase AY (0.5 g) was dissolved in the 25 mL solution which was then heated to 33° C. and agitated at 400 rpm. After a brief (10 min.) vacuum degassing, 1,111 mL of starting oil was added to the reactor. The reaction volume was overlaid with nitrogen and the reaction proceeded for 12-18 hours until all of the TAGs had been converted to fatty acids and glycerol. Once the reaction was complete, the mixture was heated to 70° C. for 1 hour to inactivate the lipase. Agitation was stopped. After the two phases had separated, the upper phase (the free fatty acid oil) was removed and stored at 4° C. in opaque plastic bottles under nitrogen.

Components of the oil samples were analyzed using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed.

### Example 3: Enzyme-modified Oil (FMO) Preparation—FFA, MAG, and DAG from Third Oil Source

Free fatty acids derived from a third oil source were prepared by the complete hydrolysis of TAGs in a starting oat oil to FFA and glycerol followed by subsequent separation of the glycerol from the FFA oil. FFA oils were prepared in a 5 L stirred, jacketed glass reactor. Citric acid buffer (100 mM, pH 5.8) was prepared by combining 24 grams of food grade citric acid with 13 g of food grade sodium hydroxide in 1,235 mL of distilled de-ionized water. Amano Lipase AY (0.5 g) was dissolved in the 25 mL solution, which was then heated to 33° C. and agitated at 400 rpm. After a brief (10 min.) vacuum degassing, 1,111 mL of starting oil was added to the reactor. The reaction volume was overlaid with nitrogen and the reaction proceeded for 12-18 hours until all of the TAGs had been converted to fatty acids and glycerol. Once the reaction was complete, the mixture was heated to 70° C. for 1 hour to inactivate the lipase. Agitation was stopped. After the two phases had separated, the upper phase (the free fatty acid oil) was removed and stored at 4° C. in opaque plastic bottles under nitrogen.

Components of the oil samples were separated using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed.

Enzyme-modified oil was produced from the FFA oil through the selective re-esterification of the FFA to glycerol to form MAG and (to a much lesser extent) DAG. FFA oil as described above was combined with 1,560 g of food grade glycerol and heated to 30° C. with agitation (300 rpm). Vacuum was applied (˜720 mmHg) to degas the material and remove residual water. Amano Lipase G (1.56 g in 50 mL DI water) was then added to the reactor and the vacuum was reapplied (˜740 mmHg). The reaction was allowed to proceed for 72 hours with progress monitored by TLC. Once the level of FFA was reduced to below 20%, the reaction was stopped by removing the vacuum, overlaying the reactor with nitrogen and heating the system to 70° C. for an hour to inactivate the enzyme.

Fatty acid concentrations were measured by titrimetry. Samples were dissolved in a solvent (95% ethanol/diethyl ether, 1/1, v/v) and titrated to neutrality (indicated with phenolphthalein) with 0.1M KOH in ethanol. Table salt (sodium chloride) was then added to the reactor. Once the salt was suspended, the agitation was stopped. After the two phases had separated (˜3 hours), the upper phase (the EMO) was removed. Antioxidant was added (tocopherol, 200 ppm) and the material was stored at 4° C. in opaque plastic bottles under nitrogen.

Analysis of EMOs was performed using an HPLC method described previously (Bruno Lima dos Santos, Kissya Kropf da Silva, Amanda Pereira Franco dos Santos, Debora França de Andrade & Luiz Antonio d'Avila (2016) Simultaneous analysis of esters and acylglycerols in biodiesel by high-performance liquid chromatography with refractive index detection, Journal of Liquid Chromatography & Related Technologies, 39:13, 620-626, DOI: 10.1080/10826076.2016.1225221he HPLC system consisted of an LC-20AD pump, SIL-20AC HT autosampler, CTO-20A column oven, and RID-10A refractive index detector (Shimadzu Scientific Instruments, Inc.). HPLC conditions were: 20 uL injection volume, isocratic elution of mobile phase consisting of 85% methanol, 10% isopropanol, and 5% hexanes, flow rate of 0.800 mL/min, and column temperature of 40° C. Column used was a SUPELCOSIL LC-18 column (250 mm×4.6 mm; 5 μm particle size). Samples were prepared in mobile phase. A mobile phase blank chromatogram was used to correct for background.

In general, the retention times were 3.4-4.0 min for glycerol, 4.0-6.2 min for MAGs and FFAs, 6.3-10.8 min for DAGs, and 11.0-50.0 min for TAGs. Integration of these peaks provided relative ratios, or area-%, of glycerol, MAG+FFA, DAG, and TAG. Since the MAG and FFA peaks overlapped, FFA content was determined by titrimetry. Samples were dissolved in a solvent (95% ethanol/diethyl ether, 1/1, v/v) and titrated to neutrality (indicated with phenolphthalein) with 0.1M KOH in ethanol. This value is reported as a weight percent. MAG content was calculated by subtracting the FFA content from the MAG+FFA determined by HPLC.

Table 1 below provides the relative glycerol, MAG, DAG, TAG, and FFA content of the resulting EMOs, which are used in the following Examples unless otherwise noted.

TABLE 1
Glycerol, MAG, DAG, TAG and FFA Content of EMOs

| | MAG | DAG | TAG | FFA |
|---|---|---|---|---|
| EMO | (area-%) | (area-%) | (area-%) | (wt-%) |
| Sunflower | 58.3 | 25.5 | BLD | 9 |
| Canola | 60.9 | 16.3 | BLD | 13.9 |
| Almond | 74.9 | 12.5 | BLD | 10 |
| Flaxseed | 57.9 | 16.5 | BLD | 18 |
| Sesame | 62.5 | 17.9 | BLD | 12 |
| Olive | 55.9 | 14.4 | BLD | 20 |
| Rosehip | 54.6 | 16.5 | BLD | 16 |
| Hemp | 69 | 11 | 2.1 | 9 |
| Oat | 65.9 | 9.4 | BLD | 13 |
| Algae | 3.2 | 28 | 32.1 | 36.6 |
| Fish | Est. 56 | Est. 14 | 15.7 | 12 |
| MCT | Est. 64 | Est. 15 | BLD | 21 |
| Coconut | Est. 72 | Est. 16 | BLD | 12 |

BLD = below limit of detection
*MAG and DAG peaks overlapped

### Example 4: Initial Screens of Broad-Spectrum Nanoemulsions

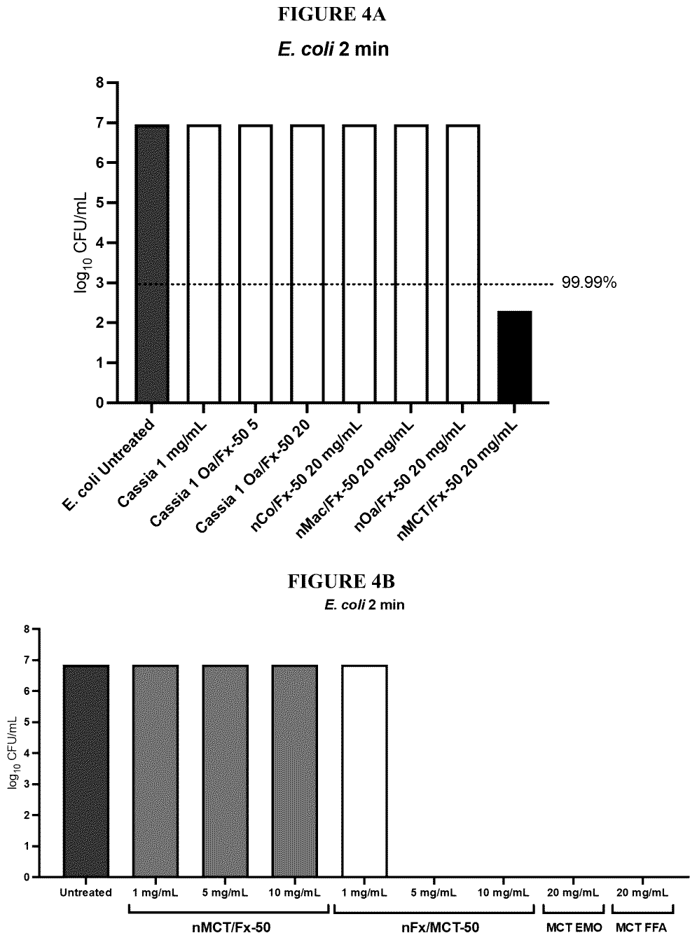

In FIG. 4A, the antimicrobial oil compositions were compared. The antimicrobial activity of the essential oil “Cassia” was compared to a combination of Cassia with a nanoemulsion of EMO, as well as a flaxseed oil derived nanoemulsion EMO, against E coli, which became the preferred bacteria to test due to ease of use and consistent growth. The amount of time was also changed to 2-minute for easier handling.

While 5 mg/mL Cassia essential oil was sufficient in a previous assay, 1 mg/mL had no effect. Combinations of 1 mg/mL Cassia and 5 or 20 mg/mL Oa/Fx-50 were also ineffective. The only nanoemulsion EMO that killed E. coli in 2-minutes was the nMCT/Fx-50 combination, with a >4-log reduction (FIG. 2, black bar).

Following this lead, nMCT/Fx-50 and nFx/MCT-50 were examined at different concentrations to determine the contribution of MCT EMO vs FFA in this context, as shown in FIG. 4B. As shown, nMCT/Fx-50 had no effect at 1-10 mg/mL, compared to its 4-log reduction at 20 mg/mL in the FIG. 4A. However, nFx/MCT-50 was completely effective at 5 mg/mL. Both of the individual components dissolved in water were effective at 20 mg/mL (MCT derived lipids are significantly more water soluble than longer chain lipids).

Without being bound by theory, the ability of MCT derived EMO and FFA as well as the Cassia essential main component cinnamaldehyde to kill E. coli at a 2-minute time interval are proposed to have the following mechanism of actions. The fastest way for molecules to enter a Gram-negative bacterial cell is through Porin-mediated diffusion, which only occurs with small hydrophilic molecules. Cinnamaldehyde, which diffuses through a porin and disrupts the electron transport chain (ETC), which in turn disrupts ATP synthesis and flagellar motor motion, result in termination of cell viability. MCT FFA and linolenic acid (one of the main components of flaxseed FFA), and the much more hydrophilic and smaller MCT FFA molecule is selectively able to diffuse through the porin where it can move toward the plasma membrane to form pores. These pores result in leakage of ATP and small proteins and disrupt a region of the cell essential for cell division, resulting in non-viability. Fighting against this movement toward the plasma membrane is the action of efflux pumps such as the RND efflux, which can remove fatty acids and other small molecules from the cell periplasm. Thus, there is a requirement to have a sufficient concentration of fatty acid to overcome this efflux and form sufficient membrane disruption in the limited timeframe of the assay.

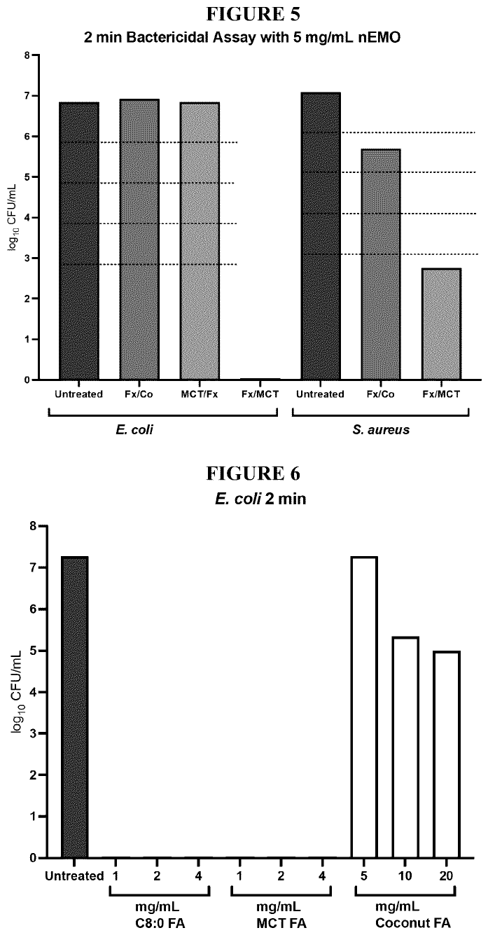

Several nanoemulsions of EMO/FFA combinations were then tested against on S. aureus (not shown) to compare activity between Gram-negative and positive bacteria to develop a product that is equally broad spectrum at a given concentration. FIG. 5 shows a summary of the general findings with 5 mg/mL nEMO/FFA. In both instances, nMCT/Fx-50 and nFx/Co-50 were insufficient in killing the bacteria in 2-minutes, while nFx/MCT-50 was sufficient. At this point, it became evident that MCT/FFA was responsible for all the observed positive effects and was further explored as an individual component. While coconut oil does contain MCTs and is the source of MCT oil, there is insufficient amount of MCT in coconut oil to provide a viable 2-min bactericidal product as shown in FIG. 6.

### Example 5: Testing of Antimicrobial Oil Compositions in a Commercial Body Wash (Dettol Body Wash, DBW)

A series of experiments were done with a commercially available body wash (Dettol). It was based on the use of the antimicrobial molecule para-chloro-meta-xylenol (PCMX, or Chloroxylenol), which was originally called Dettol. Although chloroxylenol is used body washes and liquid hand soaps, it is potentially harmful molecule for long-term use as an antimicrobial.

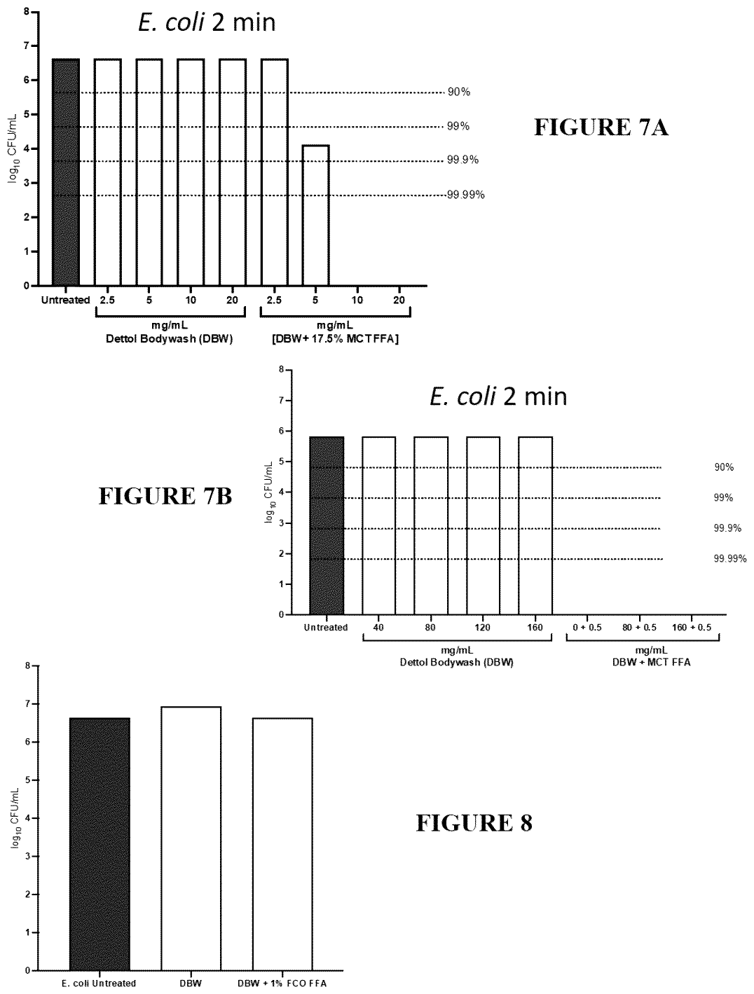

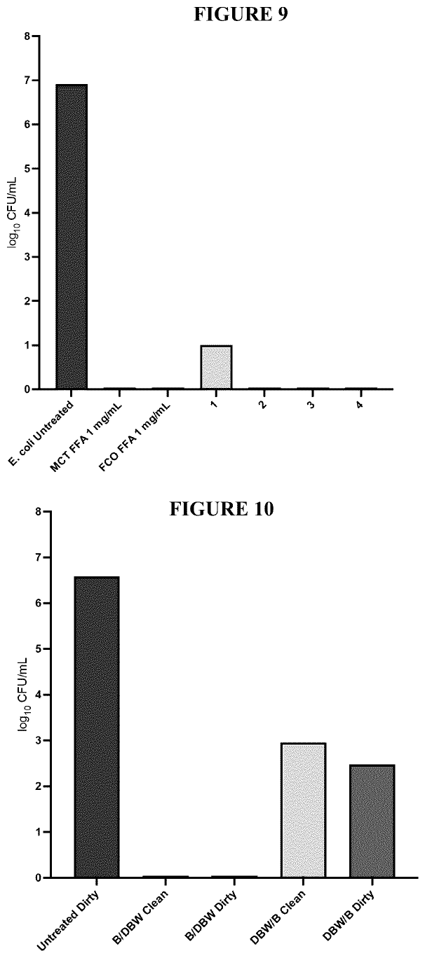

In FIGS. 7A and 7B, different concentrations of DBW alone, DBW homogenized with MCT/FFA (depicted in [ ] brackets), or DBW vortexed with MCT/FFA in the test tube were tested against E. coli in the same 2-min bactericidal assay described above. DBW had no effect on E. coli survival from 2.5 mg/mL to 160 mg/mL in phosphate buffered saline. Mixtures of DBW with MCT/FFA did kill the bacteria. [DBW+17.5% MCT/FFA] at 5 mg/mL was moderately effective, which was an MCT/FFA concentration of 0.88 mg/mL. Vortexed solutions of 0.5 mg/mL MCT/FFA with any amount of DBW were effective. This suggested that there was some matrix effect or lack of homogeneity that occurred when homogenizing DBW with MCT FFA.

A formulation with just 99% DBW and 1% FCO FFA was homogenized and tested against E. coli as shown in FIG. 8, which produced no bactericidal effect. This showed a disconnect between bactericidal activity when DBW and FFA were added into a tube and vortexed, compared to when DBW and FFA were homogenized together. For this reason, four formulations were made with a pre-homogenization step with FFA and water, with formulas shown in Table 2.

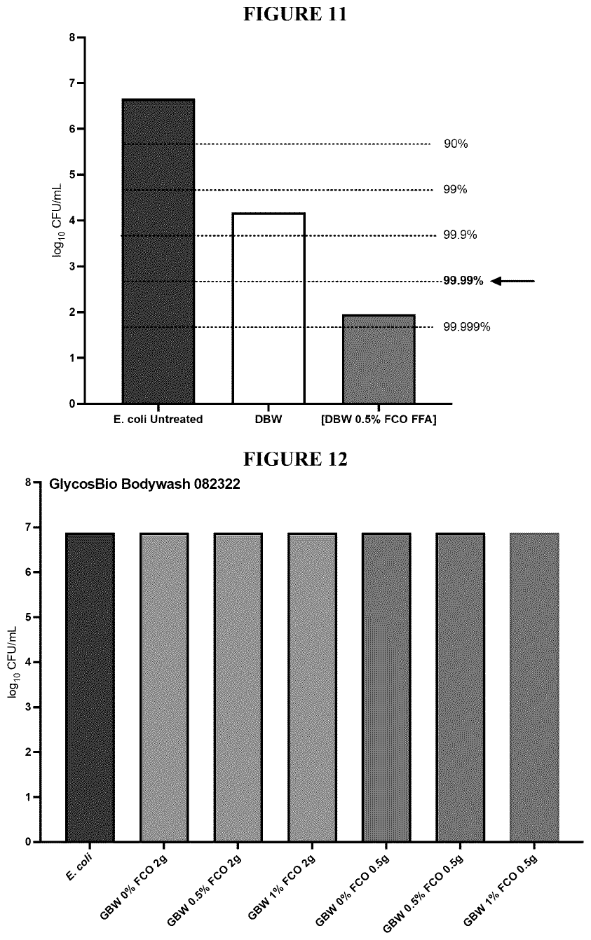

The differences in texture were most notable between the 1 and 5% oil formulations, with the 5% becoming thick and viscous compared to the flowing original material and 1% oil material. Based on the results shown in FIG. 11 there was an inability to incorporate excess oil into the DBW without first adding additional water. This suggested that the DBW was optimized specifically for the current amount of oil present.

TABLE 2
DBW formulations with MCT FFA or FCO FFA and water.

| | Type of | | | | Batch |
|---|---|---|---|---|---|
| Number | oil added | DBW | H2O | Oil | Size |
| 71922.1 | MCT FFA | 89% | 10% | 1% | 20 |
| 71922.2 | MCT FFA | 85% | 10% | 5% | 20 |
| 71922.3 | FCO FFA | 89% | 10% | 1% | 20 |
| 71922.4 | FCO FFA | 85% | 10% | 5% | 20 |

Activity was assessed using a standard industry test for antimicrobial activity. Standard test EN 1276 “Evaluation of chemical disinfectant or antiseptic for bactericidal activity” was used to assess antibacterial activity. This test consisted of 8-parts bodywash, 1-part “interfering substance,” and 1-part bacterial solution. The interfering substance for “clean conditions” was 0.3 mg/mL bovine serum albumin (BSA), and for the “dirty condition” was 3 mg/mL BSA. The reaction was conducted for 1-5 minutes and then neutralized with Dey-Engley neutralizing broth, which contained sodium bisulfite (for aldehydes), sodium thioglycolate (for mercurials), sodium thiosulfate (for iodine and chlorine), lecithin (for quaternary amines), polysorbate-80 (for phenolics), and growth nutrients for the surviving bacteria to be viable.

The test was performed with DBW in clean and dirty conditions, either by placing the interfering substance and bacterial solution on the bottom and adding the DBW on top (B/DBW), or placing DBW on the bottom of the tube and adding the interfering substance and bacterial solution on top (DBW/B). DBW/B was preferred because of the speed at which the top solution could be pipetted onto the bodywash compared to the longer amount of time it took to weigh out a bodywash on top of a liquid. Results are shown in FIG. 10.

The DBW was entirely effective in this test in the B/DBW conditions while the DBW/B left some remaining bacteria. It still produced a nearly 4-log reduction. This result showed that the DBW passed the “standard test” for antimicrobial activity.

Between the results shown for DBW in FIGS. 7A, 7B, and 8, where the bodywash was overly diluted and the results shown in FIG. 10 where the bodywash wasn't diluted enough, a middle ground was reached in FIG. 11. This allowed a modest bactericidal action of DBW (˜2.5 log reduction), while still having the bodywash be a major component of the assay at 40% of the mixture, compared to 80% in the EN 1276.

Through this modification, improvement could be measured as an additional >2-log reduction offered by the addition of FCO/FFA.

### Example 6: Testing of Antimicrobial Oil Compositions in GlycosBio Body Wash (GBW)

We built a database on ingredient compatibility using the commercial Dettol Body Wash product as a template for the ingredient list. Known antimicrobial ingredients and fragrances were not included: chloroxylenol, parfum, methylchloroisothiazolinone, and methylisothiazolinone.

The chosen ingredients are shown in Table 4, along with three initial formulation percentages. These contained 0, 0.5, and 1% FCO/FFA based on our success with dropping the AO into existing DBW, with 0% AO used a control for testing the antimicrobial capacity of the other ingredients. FIG. 12 shows that none of these formulations had sufficient AO to achieve any bactericidal action against E coli.

TABLE 3
Initial GBW formulation based on successful DBW results.

| Formulation Number | 82222.1 | 82222.2 | 82222.3 |
|---|---|---|---|
| Water | 73.1% | 72.6% | 72.1% |
| Sodium Lauryl Sulfate (SLS) | 8% | 8% | 8% |
| Glycerin | 8% | 8% | 8% |
| Cocamidopropyl Betaine | 2% | 2% | 2% |
| Propylene Glycol | 2% | 2% | 2% |
| Glycol Stearate | 2% | 2% | 2% |
| Cocamide DEA | 2% | 2% | 2% |
| Oat EMO | 2% | 2% | 2% |
| Fractionated Coconut FFA | 0% | 0.5% | 1% |
| Sodium lactate | 0.1% | 0.1% | 0.1% |
| Sodium Chloride | 0.1% | 0.1% | 0.1% |
| Sodium Citrate | 0.1% | 0.1% | 0.1% |
| Sodium Benzoate | 0.1% | 0.1% | 0.1% |
| Sodium Hydroxide | 0.1% | 0.1% | 0.1% |
| Citric Acid | 0.1% | 0.1% | 0.1% |
| Salicylic Acid | 0.1% | 0.1% | 0.1% |
| Magnesium Chloride | 0.1% | 0.1% | 0.1% |
| EDTA | 0.1% | 0.1% | 0.1% |
| Total | 100% | 100% | 100% |

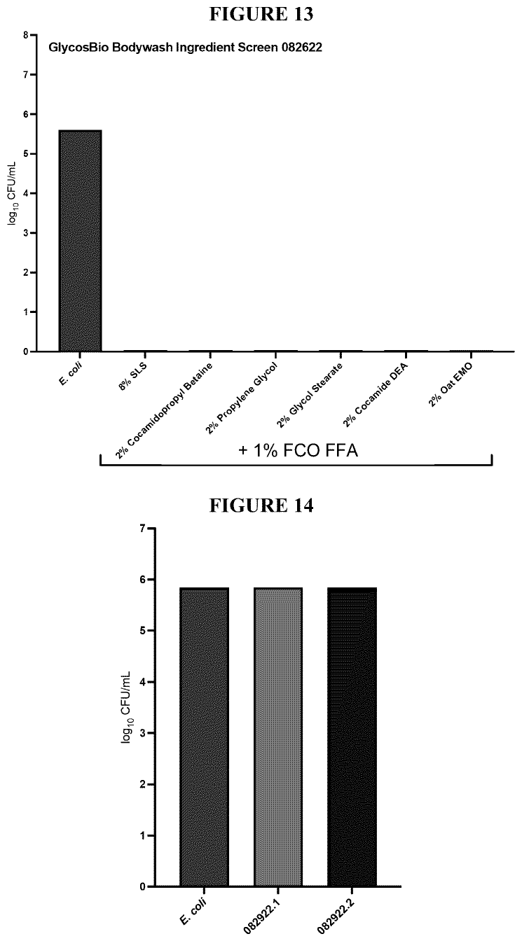

However, when the main non-salt ingredients were made into simple formulations (dissolving the same percentage of each ingredient used in the previous formulations in water with 1% FCO FFA), the 1% FCO FFA was sufficient to completely kill all E coli as shown in FIG. 13. Without being bound by theory, it was reasoned that since the main non-salt ingredients were not individually interfering, maybe one of the salts was, and since sodium lactate came as a liquid ingredient, it seemed like a place to start. Table 4 lists two formulations with or without sodium lactate and 1% FCO FFA.

TABLE 4
Formulation after ingredient screen, with/without sodium lactate.

| | 82922.1 | 82922.2 |
|---|---|---|
| Water | 73.0% | 72.9% |
| Sodium Lauryl Sulfate (SLS) | 8% | 8% |
| Glycerin | 8% | 8% |
| Cocamidopropyl Betaine | 2% | 2% |
| Propylene Glycol | 2% | 2% |
| Glycol Stearate | 2% | 2% |
| Cocamide DEA | 2% | 2% |
| Oat EMO | 2% | 2% |
| Fractionated Coconut FFA | 1% | 1% |
| Sodium lactate | 0% | 0.1% |

As shown in FIG. 14, the problem was not with the sodium lactate but instead with compatibility of FCO FFA with a combination of the ingredients.

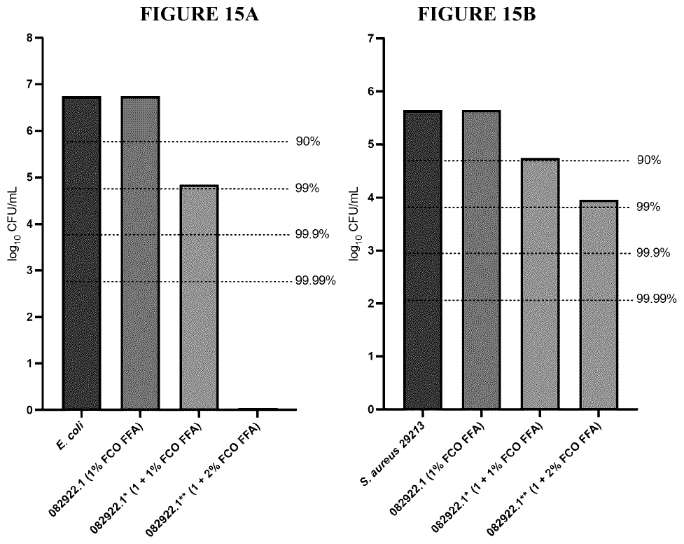

To determine whether the problem was just concentration of FCO FFA in the body wash, the 082922.1 formulation was spiked with an additional 1 or 2% FCO FFA. FIGS. 15A and 15B show that this extra material was crucial to see activity against either E. coli or S. aureus, with 3% total AO still being insufficient to achieve the 3 to 4-log reduction needed for S. aureus.

A series of body washes shown in Table 5 were then formulated, with the idea that one of the core non-salt ingredients was problematic at a high percentage but that simply lowering it would allow the FCO FFA to work at concentrations lower than the 3% required for 082922 formulations. The idea was to screen all ingredients but starting with two chemicals based on their structure: SLS and Cocamidopropyl betaine, with reductions high lighted in yellow. FCO FFA was kept at 2%.

TABLE 5
GBW ingredient screen lowering SLS and
cocamidopropyl betaine concentrations

| | 90622.0 | 90622.1 | 90622.2 | 90622.3 | 90622.4 |
|---|---|---|---|---|---|
| Water | 72% | 76% | 78% | 73% | 74% |
| Sodium Lauryl | 8% | 4% | 2% | 8% | 8% |
| Sulfate (SLS) | | | | | |
| Glycerin | 8% | 8% | 8% | 8% | 8% |
| Cocamidopropyl | 2% | 2% | 2% | 1% | 0% |
| Betaine | | | | | |
| Propylene Glycol | 2% | 2% | 2% | 2% | 2% |
| Glycol Stearate | 2% | 2% | 2% | 2% | 2% |
| Cocamide DEA | 2% | 2% | 2% | 2% | 2% |
| Oat EMO | 2% | 2% | 2% | 2% | 2% |
| Fractionated | 2% | 2% | 2% | 2% | 2% |
| Coconut FFA | | | | | |

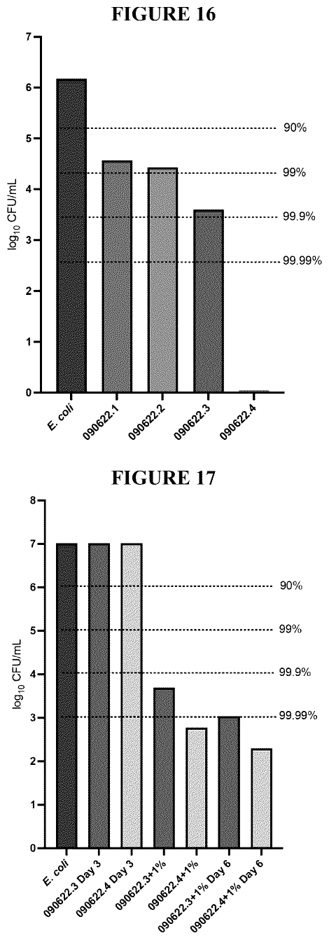

The largest change observed was from lowering the percentage of cocamidopropyl betaine as shown in FIG. 16, where lowering from 2% to 1% changed the activity from 2 to 3 logs and going from 1% to 0% allowed all bacteria to be killed.

However, when allowed to sit for 3 days, the once effective 090622.3 and 090622.4 formulations lost all activity, shown in the first three bars of FIG. 17. Addition of an additional 1% FCO FFA regained bactericidal activity, which was sustained over a 6-day period.

Several more formulations were developed as summarized in Table 6, where the SLS concentration was increased from 8 to 15%, cocamidopropyl betaine was kept at 1 or 0%, and FCO FFA was at either 2%, 5%, or introduced as a pre-homogenized blend of 1:1 FCO FFA/water (using twice as much volume for the addition). The reasoning behind the pre-homogenization was based on experience with a skin care lotion where it made a difference for activity whether the AO was directly added to the lotion/water mixture or if AO was homogenized with water to form a microemulsion before adding to the lotion.

TABLE 6
Increased SLS and FCO FFA concentrations in GBW formulations

| | 90922.1 | 90922.2 | 91222.1 | 91222.2 | 91222.3 |
|---|---|---|---|---|---|
| Water | 59% | 60% | 70% | 56% | 61% |
| Sodium Lauryl | 15% | 15% | 8% | 15% | 15% |
| Sulfate | | | | | |
| Glycerin | 15% | 15% | 8% | 15% | 15% |
| Cocamidopropyl | 1% | 0% | 1% | 1% | 1% |
| Betaine | | | | | |
| Propylene Glycol | 2% | 2% | 2% | 2% | 2% |
| Glycol Stearate | 2% | 2% | 2% | 2% | 2% |
| Cocamide DEA | 2% | 2% | 2% | 2% | 2% |
| Oat EMO | 2% | 2% | 2% | 2% | 2% |
| FCO FFA | 2% | 2% | 5% | 5% | 5% PRE-HOM |

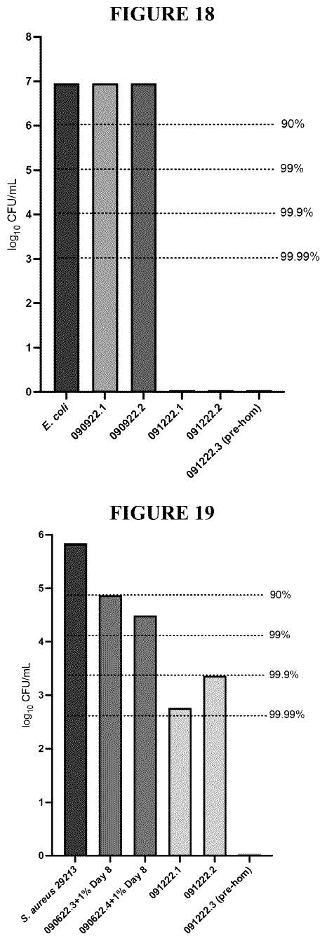

In the initial test of the formulations in FIG. 18, 090922.1 and 090922.2 with 15% SLS and 2% FCO FFA showed no antibacterial activity. The three 091222 formulations completely killed all E. coli.

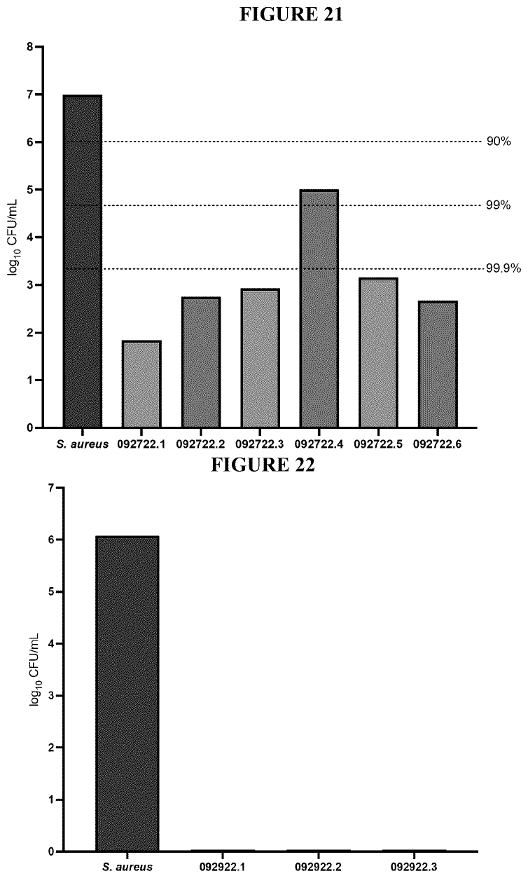

When tested against S. aureus, 090622.3 and 090622.4, which had an extra 1% FCO FFA added and had regained activity against E. coli in FIG. 17, had lower activity of 1 to 1.5-log reduction (blue bars in FIG. 21). The 091222 formulas were all active, with a marked improvement in the pre-homogenized 091222.3. All formulations after this one were done with the pre-homogenization approach.

Next a complete version of the GlycosBio Body Wash (GBW) was formulated, using 15% SLS, 5% FCO FFA, and all of the salts, shown in Table 7.

TABLE 7
GBW formulation with increased SLS/FFA and all salts

| | 91622.1 |
|---|---|
| Water | 55.1% |
| Sodium Lauryl Sulfate | 15% |
| Glycerin | 15% |
| Cocamidopropyl Betaine | 1% |
| Propylene Glycol | 2% |
| Glycol Stearate | 2% |
| Cocamide DEA | 2% |
| Oat EMO | 2% |
| FCO FFA | 5% |
| Sodium lactate | 0.1% |
| Sodium Chloride | 0.1% |
| Sodium Citrate | 0.1% |
| Sodium Benzoate | 0.1% |
| Sodium Hydroxide | 0.1% |
| Citric Acid | 0.1% |
| Salicylic Acid | 0.1% |
| Magnesium Chloride | 0.1% |
| EDTA | 0.1% |

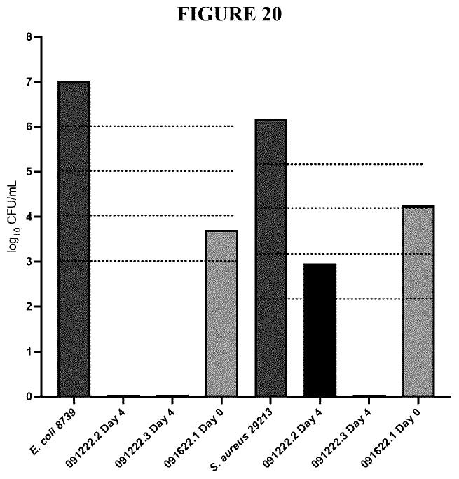

FIG. 20 shows that the previous formulations from 091222.2 and 091222.3 at Day 4 were still effective against E. coli and S. aureus, but the addition of salts in 091622.1 gave a less potent effect particularly against S. aureus where only a 2-log reduction was observed.

It was reasoned that one or more of the salts might be having a detrimental effect, so individual salts were removed from the formulation as shown in Table 8. The plan was to test the removal of all of them, but sodium lactate, sodium chloride, salicylic acid, magnesium chloride, and EDTA were chosen first with the idea that common pH modifiers like citric acid and sodium hydroxide would have less of an influence. It was also decided to try 12% SLS/glycerin in these formulations to reduce the thickness, since the 15% SLS formulations would become more like creams in some instances.

TABLE 8
GBW formulations with selected salts removed.

| | 92222.1 | 92222.2 | 92222.3 | 92222.4 | 92222.5 | 92222.6 |
|---|---|---|---|---|---|---|
| Water | 61.1% | 61.2% | 61.2% | 61.2% | 61.2% | 61.2% |
| Sodium Lauryl Sulfate | 12% | 12% | 12% | 12% | 12% | 12% |
| Glycerin | 12% | 12% | 12% | 12% | 12% | 12% |
| Cocamidopropyl Betaine | 1.0% | 1% | 1.0% | 1.0% | 1.0% | 1.0% |
| Propylene Glycol | 2% | 2% | 2% | 2% | 2% | 2% |
| Glycol Stearate | 2% | 2% | 2% | 2% | 2% | 2% |
| Cocamide DEA | 2% | 2% | 2% | 2% | 2% | 2% |
| Oat EMO | 2% | 2% | 2% | 2% | 2% | 2% |
| FCO FFA | 5% | 5% | 5% | 5% | 5% | 5% |
| Sodium lactate | 0.1% | — | 0.1% | 0.1% | 0.1% | 0.1% |
| Sodium Chloride | 0.1% | 0.1% | — | 0.1% | 0.1% | 0.1% |
| Sodium Citrate | 0.1% | 0.1% | 0.1% | 0.1% | 0.1% | 0.1% |
| Sodium Benzoate | 0.1% | 0.1% | 0.1% | 0.1% | 0.1% | 0.1% |
| Sodium Hydroxide | 0.1% | 0.1% | 0.1% | 0.1% | 0.1% | 0.1% |
| Citric Acid | 0.1% | 0.1% | 0.1% | 0.1% | 0.1% | 0.1% |
| Salicylic Acid | 0.1% | 0.1% | 0.1% | — | 0.1% | 0.1% |
| Magnesium Chloride | 0.1% | 0.1% | 0.1% | 0.1% | — | 0.1% |
| EDTA | 0.1% | 0.1% | 0.1% | 0.1% | 0.1% | — |
| pH: | 5.49 | 5.53 | 5.48 | 6.15 | 5.82 | 5.53 |

The results shown in FIG. 21 (against S. aureus) point to the absence of salicylic acid (092222.4) as a significant factor in reducing the antimicrobial activity. After the fact, pHs were measured of each of these formulations, which are listed in Table 7, and 092222.4 with salicylic acid removed had a pH value of 6.15 which was more than half a unit higher than some of the others. This is likely the reason for its failure. Nevertheless, most of these formulations achieved acceptable germ kill levels. The influence of pH on activity is described later.

Two potential failure points were addressed: increasing the pH of a working formula to 6 or higher results in lowering the amount of AO in this new formulation setting. 092922.1 was formulated as usual, and 1M sodium hydroxide was added until reaching a pH of 6. 092922.2 and 092922.3 have 1% Oat EMO and either 4 or 3% FCO FFA, shown in Table 9.

TABLE 9
Raising pH of effective formulation,
lowering amount of AO in formulations

| | 92922.1 | 92922.2 | 92922.3 |
|---|---|---|---|
| Water | 61.1% | 63.1% | 64.1% |
| Sodium Lauryl Sulfate | 12% | 12% | 12% |
| Glycerin | 12% | 12% | 12% |
| Cocamidopropyl Betaine | 1% | 1% | 1% |
| Propylene Glycol | 2% | 2% | 2% |
| Glycol Stearate | 2% | 2% | 2% |
| Cocamide DEA | 2% | 2% | 2% |
| Oat EMO | 2% | 1% | 1% |
| FCO FFA | 5% | 0.1% | 0.1% |
| Sodium lactate | 0.1% | 0.1% | 0.1% |
| Sodium Chloride | 0.1% | 0.1% | 0.1% |
| Sodium Citrate | 0.1% | 0.1% | 0.1% |
| Sodium Benzoate | 0.1% | 0.1% | 0.1% |
| Sodium Hydroxide | 0.1% | 0.1% | 0.1% |
| Citric Acid | 0.1% | 0.1% | 0.1% |
| Salicylic Acid | 0.1% | 0.1% | 0.1% |
| Magnesium Chloride | 0.1% | 0.1% | 0.1% |
| EDTA | 0.1% | 5.52 | 5.45 |
| pH: | 6.00 | | |

In this case, the differences were uninformative because all 3 formulations completely eradicated S. aureus, shown in FIG. 22.

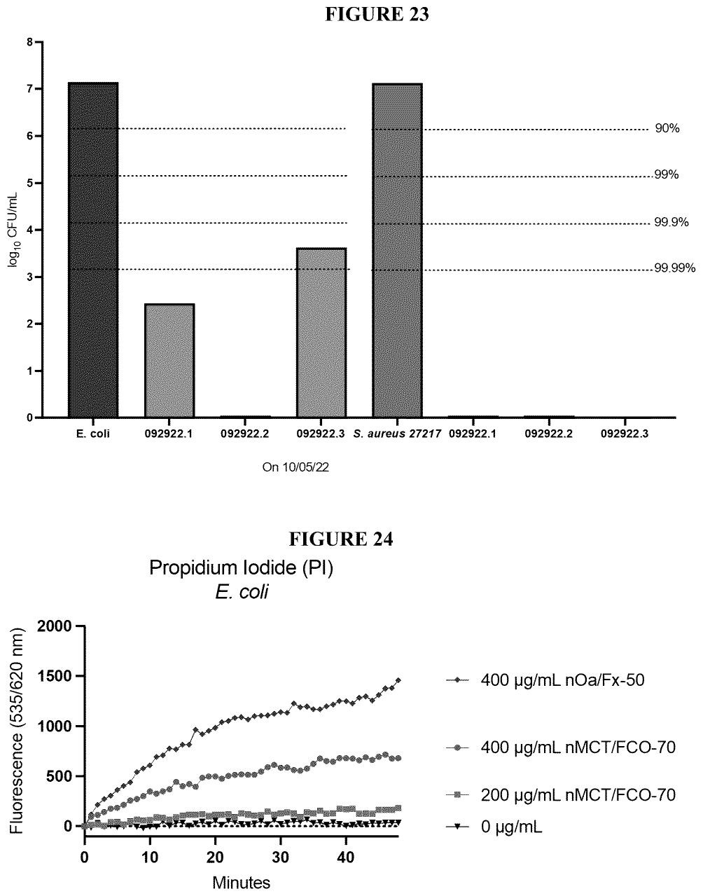

After one week, the formulas from Table 9 were retested against E. coli and S. aureus (FIGS. 23A and 23B). A different strain of S. aureus was used because on-skin experiment were also planned. The less pathogenic strain S. aureus ATCC 27217 was used. This strain was much more susceptible to FCO FFA since all of the week-old formulations were completely effective. The formulations were effective against E coli as well, but 092922.3 was borderline with only a 3.5-log reduction.

### Example 7. Mechanistic Studies of FCO FFA

Without being bound by theory, it is believed that because FCO FFA and other saturated medium chain fatty acids are functional against E coli because they possess much greater polarity than longer chain or unsaturated oils and can likely get privileged quick access to the bacterial cell via porin channels. To get a better understanding of which processes are involved in the action, three commonly used dyes were used to probe the various aspects of membrane equilibrium. Nanoemulsions were made that contained mostly FCO FFA so that they would be stable and water-soluble for the duration of the experiment. Nanoemulsions shown in Table 10 were made as 1:9 oil in water emulsions that were sonicated for 5 minutes to achieve sizes of 164-252 nm as measured by DLS. 081522.1 was 100% FCO FFA as the oil phase and had the largest size, while combinations of MCT EMO from step 2 and FCO FFA in 10:90 or 30:70 (081522.2 and 081522.3 respectively) gave smaller sizes. All three nanoemulsions increased in size at the 24-hr time point, with the nMCT/FCO-70 being an acceptable 211.1 nm. This nAO was used for the dye experiments.

TABLE 10
Nanoemulsions with FCO FFA: formulations and DLS data at 0 and 24 hrs

| | | | | | | | Time 24 | |
|---|---|---|---|---|---|---|---|---|
| | | FCO | FCO | | Time 0 | | hr | |
| | Code | EMO | FFA | H2O | Z-ave | PDI | Z-ave | PDI |
| 81522.1 | nFCO-100 | 0 g | 3 g | 27 g | 252.4 | 0.25 | 311 | 0.221 |
| 81522.2 | nMCT/FCO-90 | 0.3 g | 2.7 g | 27 g | 218.5 | 0.227 | 255.2 | 0.242 |
| 81522.3 | nMCT/FCO-70 | 0.9 g | 2.1 g | 27 g | 164.2 | 0.26 | 211.1 | 0.184 |

The membrane-impermeable fluorescent DNA-intercalating dye propidium iodide was used to assay the effect of nanoemulsion nMCT/FCO-70. An overnight culture of E. coli was pelleted, washed twice with buffer (10 mM HEPES buffer, pH 7.4, 50 μg/mL CaCl₂), 5 mM glucose), resuspended in buffer, and added to a 96-well plate containing nMCT/FCO-70 or nOa/Fx-50 at a final concentration of 0, 100, 200, or 400 μg/mL in triplicate. The plate was incubated at 37° C. for 10 min, then 5 μg/mL of propidium iodide was added and immediately put on a Biotek Cytation 5 plate reader to read fluorescence every minute for 90 minutes (ex/em: 535/620 nm). Background fluorescence from untreated wells was subtracted from all readings.

FIG. 24 shows that 400 μg/mL of nOa/Fx-50 produced a higher fluorescent signal than an equivalent amount of nMCT/FCO-70, suggesting that membrane permeability was more influenced by unsaturated long chain oils.

The membrane potential-sensitive fluorescent dyes 3,3-dipropylthiacarbocyanide DiSC3(5) and DiOC2 were used to assay the effect of nMCT/FCO-70. An overnight culture tube of E. coli was pelleted, washed once with buffer (5 mM HEPES pH 7.2, 100 mM KCl, 20 mM glucose), resuspended in buffer to an OD600=0.1, and DiSC3(5) or DiOC2 was added to the tube at a final concentration of 100 nM. The tube was wrapped in foil and shaken at 220 rpm in a 37° C. incubator for 30 minutes. In a 96-well black polystyrene plate, 16 μL of nanoemulsion or water, and 184 μL of dye-saturated cells were added to make final nanoemulsion concentrations of 0, 100, 200 or 400 μg/mL in triplicate. The plate was immediately placed on a Biotek Cytation 5 plate reader to read fluorescence every 10 s for 60 min (ex/em: 622/670 nm).

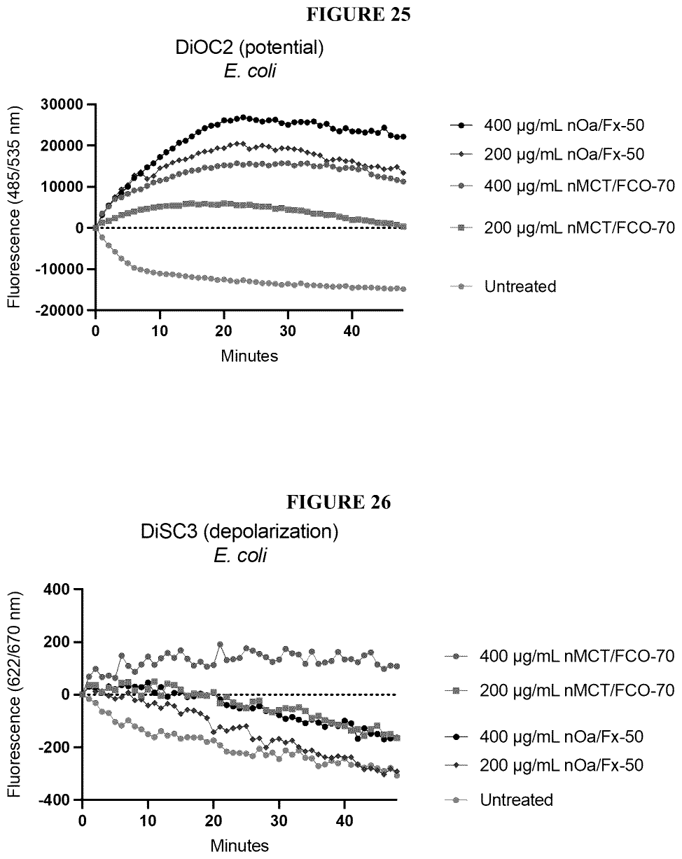

FIG. 25 shows that nOa/Fx-50 elicits a much larger fluorescent signal than nMCT/FCO-70, indicating that membrane potential is more influenced by unsaturated long chain oils such as oat and flaxseed.

In FIG. 26, there was a reversal of the trend with nMCT/FCO-70 generating more fluorescence from DiSC3 than nOa/Fx-50. This suggested that short chain saturated fatty acids increase sodium ion permeability, which would produce a greater depolarization, making the membrane less negatively charged. Since treatment of bacteria with FCO FFA resulted in very quick (1-2 minute) killing, it seems that a mechanism that focused on depolarization and its downstream effects is a quicker mechanism of action relative to the longer chain unsaturated oils that required several minutes-hours and influence more the membrane potential and permeability.

### Example 8: Effects of pH

The goal of the following experiments was to identify the working pH range of the Activated Oil (AO), in particular the short chain fatty acids such as those found in FCO FFA.

FIG. 27 compares the structure of benzoic acid—sodium benzoate to fatty acid—fatty acid salts at acidic versus basic pH. It is commonly known that benzoic acid has a strong working antimicrobial range of pH 2.5 to 4 and is not recommended as a preservative at pH higher than 4.5, due to the increasing concentration of sodium benzoate which is weakly antimicrobial.

The antimicrobial activity of fatty acids is pH-dependent, chain length-dependent, and unsaturation-dependent, as well as whether they are modified as glyceryl-, sugar-, or hydrocarbon esters (see review: McGaw et. al., South African J of Botany, 2002).

Short chain fatty acids such as caprylic acid are generally more bactericidal at lower pHs where the carboxylic acid is protonated. However, the massively increased water-solubility of the salt-form sodium caprylate at higher pHs accounts can account for increased access to bacteria and entry into cells. This balance of activity and accessibility has resulted in unexpected findings and broad applications to certain use-cases including hygiene products to include bodywashes, bar soaps, and other cleansers.

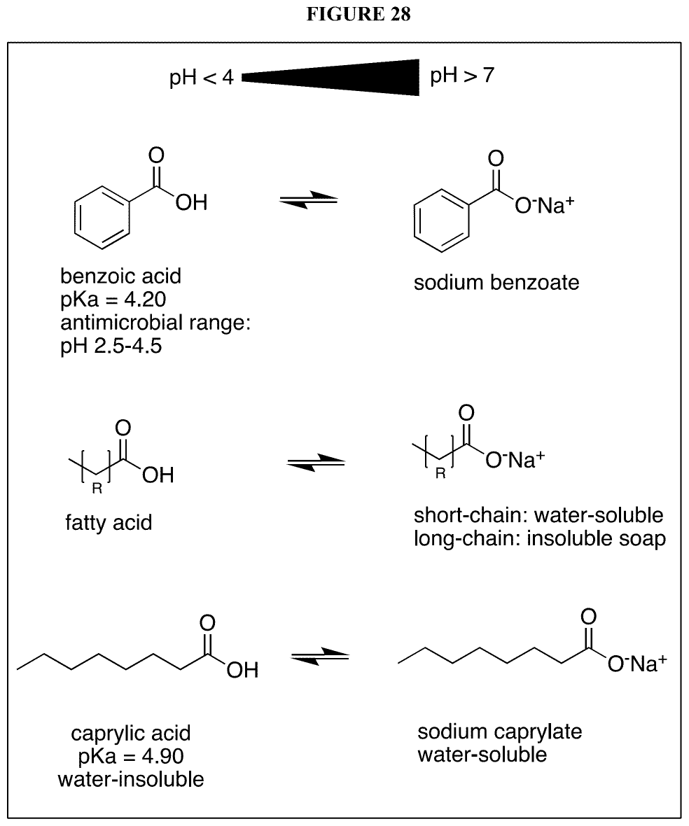

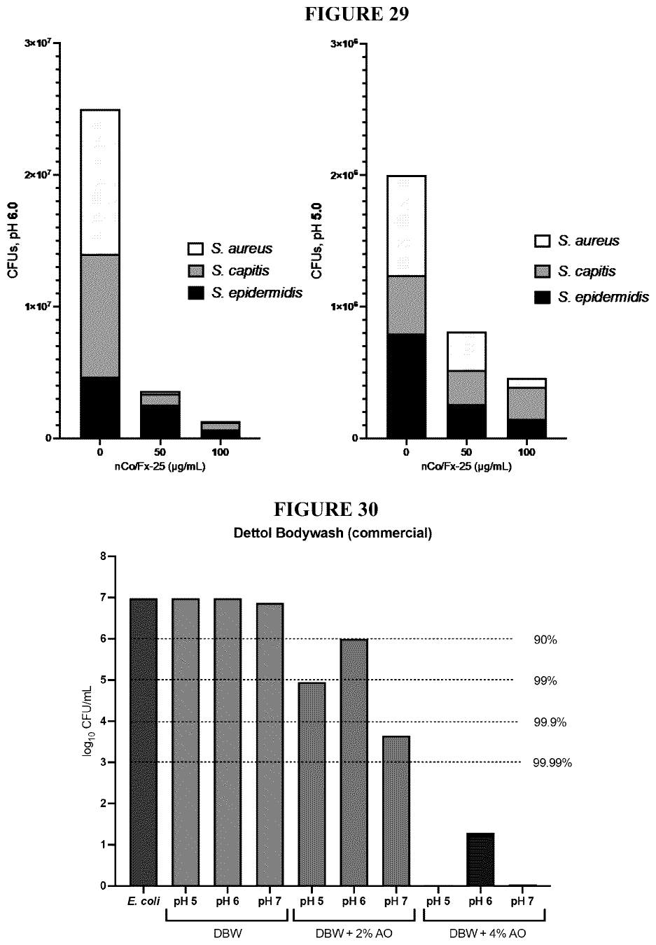

FIG. 28 show how three Staphylococcal species: S. aureus, S. capitis, and S. epidermidis were cultured separately and mixed into a single test-tube and treated with a nanoemulsion containing Coconut EMO (25%) and Flaxseed FFA (75%) at 50 and 100 μg/mL at various pHs, with the surviving plated species quantified. Overall survival of species in the untreated samples was about a log-unit lower at pH 5.0 (2×10\hat{ }6 CFU) than at pH 6.0 (2.5×10\hat{ } 7 CFU) and a great degree of the anti-S. aureus selective activity (reduction of white bar) shown at pH 6.0 is not found at pH 5.0. S. aureus is nearly undetectable when treated with this particular nanoemulsion at pH 6.0 but is still a major component of the 3-species mix after nanoemulsion treatment at pH 5.0.

### Example 9. PH-Related Experiments with a Commercial Bodywash

A commercial bottle of Dettol “original antibacterial pH-balanced bodywash” was purchased and used for the following experiments. A 2N NaOH solution was used for adjusting pH of the bodywashes. The ingredients in the Dettol we used were: Water, Sodium Lauryl Sulfate, Glycerin, Cocamidopropyl Betaine, Sodium Chloride, Glycol Stearate, Propylene Glycol, Cocamide DEA, Sodium Citrate*, Citric Acid*, Salicylic Acid*, Parfum, Chloroxylenol*, Tetrasodium EDTA*, Sodium Lactate*, Sodium Benzoate*, Magnesium Nitrate*, Sodium Hydroxide, Magnesium Chloride, Methylchloroisothiazolinone*, Methylisothiazolinone*.

DBW as 80% bodywash in the assay (4 g bodywash, 0.5 g PBS, 0.5 g bacteria in PBS) resulted in complete germ-kill of E. coli and S. aureus, but diluting to 50% bodywash in the assay (2.5 g bodywash, 2.0 g PBS, 0.5 g bacteria in PBS) is enough to weaken the activity of DBW against E coli. In this case, the 50% bodywash was used in a 1-min bactericidal assay at 37° C. with immediate neutralization with Dey-Engley Neutralizing Broth.

As shown in FIG. 30, no germ-kill was observed with DBW, DBW+2% antioxidant oils (AO) had mixed results in which a 2-log reduction was observed at pH 5, only a 1-log reduction at pH 6, and a 3.5-log reduction at pH 7.2% AO addition at pH 6 may be a complicated dynamic of failing DBW antibacterial components, but sub-optimal water-solubility of the AO fatty acids, which is then overcome at pH 7.4% AO addition was sufficient to achieve complete or near-complete germ-kill at all tested pHs.

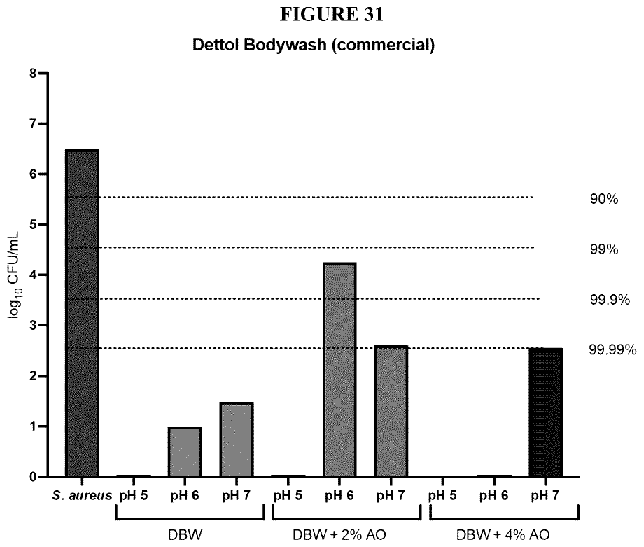

Shown in FIG. 31, the DBW product was very effective against S. aureus (ATCC 6538) and only had a slight reduction in activity at pH 6 and 7. There appeared to be some interference of the DBW antimicrobials at pH 6 when 2% AO was incorporated, and this interference was reduced or overcome at pH 7.

1.-20. (canceled)

21. An oil composition comprising:
a first oil component, the first oil component comprising free fatty acids of a fractionated coconut oil, wherein the free fatty acids of the fractionated coconut oil comprise at least 99% by weight of the total weight of the first oil component, and wherein the first oil component comprises at least 20% by weight of the total weight of the oil composition;
and a second oil component, the second oil component comprising free fatty acids of an oil source comprising a fatty acid profile that is different from the fractionated coconut oil, wherein the second oil component comprises at least 10% by weight of the total weight of the oil composition.

22. The oil composition of claim 21, wherein the oil source is rosehip oil.

23. The oil composition of claim 22, wherein the first oil component comprises at least 70% by weight of the total weight of the oil composition.

24. The oil composition of claim 21, wherein the second oil component further comprises a mixture of diacylglycerides and monoacylglycerides.

25. The oil composition of claim 24, wherein the mixture of diacylglycerides and monoacylglycerides comprise at least 20% by weight of the total weight of the second oil component.

26. The oil composition of claim 21, wherein the second oil component comprises at least 70% by weight of the total weight of the oil composition.

27. The oil composition of claim 26, wherein the oil source is rosehip oil.

28. The oil composition of claim 21, wherein the free fatty acids of the oil source comprise at least 99% by weight of the total weight of the second oil component.

29. The oil composition of claim 21, wherein the oil source is rosehip oil, wherein the first oil component comprises 75% by weight of the total weight of the oil composition, wherein the second oil component comprises 25% by weight of the total weight of the oil composition, and wherein the free fatty acids of the oil source comprise at least 99% by weight of the total weight of the second oil component.

30. The oil composition of claim 21, wherein the oil source is rosehip oil, wherein the first oil component comprises 25% by weight of the total weight of the oil composition, wherein the second oil component comprises 75% by weight of the total weight of the oil composition, and wherein the free fatty acids of the oil source comprise at least 99% by weight of the total weight of the second oil component.

31. The oil composition of claim 21 further comprising a third oil component, wherein the third oil component comprises a mixture of monoacylglycerides, diacylglycerides, and free fatty acids derived from rosehip oil, wherein the third oil component is substantially free from triglycerides, wherein the oil source is rosehip oil, and wherein the free fatty acids of the oil source comprise at least 99% by weight of the total weight of the second oil component.

32. The oil composition of claim 31, wherein the first oil component comprises at least 70% by weight of the total weight of the oil composition, wherein the third oil component comprises at least 10% by weight of the total weight of the oil composition.

33. A composition comprising:
a surfactant; and an oil composition, wherein the oil composition is in an amount of about 0.25% to about 10% by weight of the total weight of the composition, the oil composition comprising a first oil component, the first oil component comprising free fatty acids of a fractionated coconut oil, wherein the free fatty acids of the fractionated coconut oil comprise at least 99% by weight of the total weight of the first oil component, and
wherein the first oil component comprises at least 20% by weight of the total weight of the oil composition, and a second oil component, the second oil component comprising free fatty acids of an oil source comprising a fatty acid profile that is different from the fractionated coconut oil, wherein the second oil component comprises at least 10% by weight of the total weight of the oil composition.

34. The composition of claim 33, wherein the oil source is rosehip oil.

35. The composition of claim 34, wherein the first oil component comprises at least 70% by weight of the total weight of the oil composition.

36. The composition of claim 33, wherein the second oil component further comprises a mixture of diacylglycerides and monoacylglycerides.

37. The composition of claim 36, wherein the mixture of diacylglycerides and monoacylglycerides comprise at least 20% by weight of the total weight of the second oil component.

38. The composition of claim 33, wherein the second oil component comprises at least 70% by weight of the total weight of the oil composition.

39. The composition of claim 38, wherein the oil source is rosehip oil.

40. The composition of claim 33, wherein the free fatty acids of the oil source comprise at least 99% by weight of the total weight of the second oil component.
